(19) United States
(12) Patent Application Publication
(10) Pub. No.: US 2022/0001081 A1
(43) Pub. Date: Jan. 6, 2022

(54) SURFACE TREATED BIOCOMPOSITE MATERIAL, MEDICAL IMPLANTS COMPRISING SAME AND METHODS OF TREATMENT THEREOF

(71) Applicant: Ossio Ltd., Caesarea (IL)

(72) Inventors: Orahn PREISS-BLOOM, Zichron Yakov (IL); Taly Pnina LINDNER, Savyon (IL); Ilan UCHITEL, Kfar-Saba (IL); Tal ZEEVI, Pardes Hana-Karkur (IL)

(21) Appl. No.: 17/268,449

(22) PCT Filed: Jul. 25, 2019
(86) PCT No.: PCT/IL2019/050843

§ 371 Date: Feb. 12, 2021

Related U.S. Application Data
(60) Provisional application No. 62/723,158, filed on Aug. 27, 2018.

(51) Int. Cl. A61L 27/58; A61F 2/30; C08J 5/04; A61L 27/46; C08L 67/04

(52) U.S. Cl. CPC A61L 27/58; A61F 2/30767; C08J 5/0405; A61L 27/46; C08J 2367/04; A61L 2430/02; A61L 2400/12; A61L 2400/18; A61L 2430/38; C08L 67/04

(57) ABSTRACT

Reinforced biocomposite materials having a unique treated surface, in which the surface may comprise a plurality of layers. According to at least some embodiments, medical implants are provided that incorporate novel structures, alignments, orientations and forms comprised of such reinforced bioabsorbable materials, as well as methods of treatment thereof.

### FIELD OF THE INVENTION

The present invention is of a precision surface treated biocomposite material, medical implants comprising same and methods of treatment thereof, and in particular to such material, implants and methods of treatment that have medical applications.

### BACKGROUND OF THE INVENTION

The mechanical strength and modulus (approximately 3-5 GPa) of non-reinforced resorbable polymers is insufficient to support fractured cortical bone, which has an elastic modulus in the range of approximately 15-20 GPa. For example, in an article the bending modulus of human tibial bone was measured to be about 17.5 GPa (Snyder S M Schneider E, Journal of Orthopedic Research, Vol. 9, 1991, pp. 422-431). Therefore, the indications of existing medical implants constructed from resorbable polymers are limited and their fixation usually requires protection from motion or significant loading. These devices are currently only a consideration when fixation of low stress areas is needed (i.e. non-load bearing applications) such as in pediatric patients or in medial malleolar fractures, syndesmotic fixation, maxillofacial, or osteochondral fractures in adults.

A new class of reinforced composite biomaterials (biocomposites) has been recently introduced wherein a bioabsorbable and biocompatible polymer is reinforced by bioabsorbable, biocompatible glass fibers. These materials can achieve improved mechanical properties. These materials also involve a compatibilizer to bind the polymer to the reinforcing fibers. Examples of such materials are described in the following two patent applications, which are included fully herein by reference as if fully set forth herein:

1. Biocompatible composite and its use (WO2010122098)

2. Resorbable and biocompatible fiber glass compositions and their uses (WO2010122019)

These materials have been further described and characterized in publications associated with these patents including
1. Lehtonen T J et al. Acta Biomaterialia 9 (2013) 4868-4877
2. Lehtonen T J et al. J Mech Behavior BioMed Materials. 20 (2013) 376-386

The development of this class of materials described in the background art has focused on the composition of the materials: the bioabsorbable polymer, the reinforcing mineral fiber, the compatibilizer, and the combinations between them. These compositions have been demonstrated to be capable of achieving mechanical properties superior to the mechanical properties previously achieved with bioabsorbable polymers alone.

However, while material composition is one parameter that can affect mechanical properties of a medical implant, when it comes to composite materials, the material composition does not by itself ensure mechanical properties that are sufficient for the implant to achieve its desired biomechanical function. In fact, reinforced composite medical implants with identical compositions and identical geometries can have vastly different mechanical properties. Furthermore, even within the same implant, mechanical properties can vary greatly between different mechanical axes and between different types of mechanical strength measurements.

### SUMMARY OF THE INVENTION

The background art does not teach or suggest biocomposite materials that have one or more desirable mechanical characteristics. The background art also does not teach or suggest such materials that can achieve a desired biomechanical function.

By “biocomposite material” it is meant a composite material that is biologically compatible or suitable, and/or which can be brought into contact with biological tissues and/or which can be implanted into biological materials and/or which will degrade, resorb or absorb following such implantation.

By “biocompatible” it is meant a material that is biologically compatible or suitable, and/or which can be brought into contact with biological tissues, and/or which can be implanted into biological materials.

By “surface treated” biocomposite material it is meant a material which features at least a surface layer, and optionally a plurality of surface layers.

The present invention, in at least some embodiments, relates to surface treated biocomposite materials which overcome the drawbacks of the background art. According to at least some embodiments, medical implants are provided that incorporate novel structures, alignments, orientations and forms comprised of such surface treated bioabsorbable materials.

The surface treated bioabsorbable materials are implemented as a biomedical implant, featuring a body composition and a surface layer, also described herein as a “surface”. The body composition preferably features a combination of mineral and polymer, while the surface optionally features a different composition than the implant body. The surface may optionally comprise a plurality of surface layers, including at least one outer surface layer and at least one inner surface layer.

Optionally, 10-70% w/w of the body composition comprises mineral material. Also optionally, 30-55% w/w of the body composition comprises mineral material. Also optionally, 45-55% w/w of the body composition comprises mineral material.

Optionally, the polymer comprises PLDLA. Also optionally mineral material of said body composition comprises ranges of the following elements, all mol %: Na2O: 11.0-19.0, CaO: 9.0-14.0, MgO: 1.5-8.0, B2O3: 0.5-3.0, Al₂O₃: 0-0.8, P2O3: 0.1-0.8, SiO2: 67-73. Optionally, the mineral material of said body composition comprises ranges of the following elements, all mol %: Na2O: 12.0-13.0 mol. %, CaO: 9.0-10.0 mol. %, MgO: 7.0-8.0 mol. % B2O3: 1.4-2.0 mol. % P2O3: 0.5-0.8 mol. %, SiO2: 68-70 mol. %. Optionally, the mineral material of said body composition comprises ranges of the following elements, all mol %: Na2O: 11.0-19.0, CaO: 8.0-14.0, MgO: 2-8.0, B2O3: 1-3.0, Al₂O₃: 0-0.5, P2O3: 1-2, SiO2: 66-70% mol %.

The surface layer may optionally be implemented with a variety of different portions of the surface area having a different composition than the body composition. For example and without limitation, optionally more than 10% of an area of the surface is of a different composition than the body. Optionally more than 30% of the surface area is of a different composition than the body. Optionally more than 50% of the surface area is of a different composition than the body.

Optionally the body comprises an interspersed composition of mineral and polymer.

Optionally the surface layer may have various thicknesses, of which various non-limiting examples are given herein. For example, optionally the surface layer is defined as an outer layer of up to 100 micron depth. Optionally said outer layer is up to 50 micron depth. Optionally said outer layer is up to 20 micron depth. Optionally said outer layer is up to 5 micron depth. Optionally the surface layer is a uniform polymer or a bio composite composition different than the internal composition.

The surface layer may also optionally feature various concentrations of various materials. For example, optionally the surface layer of up to 5 microns includes an increase phosphate concentration. Optionally said outer layer features said increase in phosphate to more than 10% w/w. Optionally the outer layer includes an increase in calcium to more than five times the body composition. Optionally said increase in calcium is more than ten times the body composition. Optionally said increase in calcium is more than fifteen times the body composition.

Various surface layer structures are also optionally possible. For example, optionally said surface layer comprises a plurality of separate distinguished layers, each comprising a different composition.

Optionally the surface layer comprises at least an inner layer and an outer layer, wherein the outer layer is up to 3 microns and features an increase in phosphate to more than 5% w/w, wherein the inner layer is up to 20 microns and does not feature phosphate.

Optionally the treated portion of the surface layer has a roughness increase by more than five times the untreated surface. Optionally the treated portion of the surface layer has a roughness increase by more than ten times the untreated surface. Optionally the treated surface area increases the surface area by more than 15%. Optionally the treated surface area increases the surface area by more than 50%. Optionally the surface has a different mineral composition.

The body composition may also optionally feature various amounts of minerals and other ingredients. For example, optionally the body composition comprises more than 20% w/w mineral. Optionally the body composition comprises more than 40% w/w mineral.

Optionally said implant is cannulated, said surface layer comprises an inner surface layer and an outer surface layer, and an inner surface layer composition is different than an outer surface layer composition.

Optionally said implant is cannulated, said surface layer comprises an inner surface layer and an outer surface layer, and an inner surface layer composition is the same as an outer surface layer composition.

Optionally a surface of said implant is treated to partially expose inner composition.

Optionally the surface maximum roughness is more than 2 microns.

Optionally the surface maximum roughness is more than 3 microns.

Optionally said body composition comprises more than 8% w/w silica but said surface composition comprises less than 4% w/w silica.

Optionally the implant comprises a plurality of holes. Optionally said holes comprise an inner surface that is different from said surface of the implant. Optionally said inner surface comprises a different composition. Optionally said holes comprise an inner surface comprising a composition of said surface of the implant.

Optionally the body composition may be implemented as a plurality of reinforcing fibers, also as described herein.

These medical implants have unique mechanical properties. They have great clinical benefit in that these implants can have mechanical properties that are significant greater than those of the currently available bioabsorbable polymer implants. The term “mechanical properties” as described herein may optionally include one or more of elastic modulus, tensile modulus, compression modulus, shear modulus, bending moment, moment of inertia, bending strength, torsion strength, shear strength, impact strength, compressive strength and/or tensile strength.

Optionally any of the embodiments or sub-embodiments as described herein may be combined, for example in regard to any of the implant properties, implant structures or implant surface treatments, or any combination of any aspect of the same.

Without wishing to be limited by a closed list or a single hypothesis, the biocomposite implants described herein represent a significant benefit over metal or other permanent implants (including non absorbable polymer and reinforced polymer or composite implants) in that they are absorbable by the body of the subject receiving same, and thus the implant is expected to degrade in the body following implantation. Again without wishing to be limited by a closed list or a single hypothesis, they also represent a significant benefit over prior absorbable implants since they are stronger and stiffer than non-reinforced absorbable polymer implants in at least one mechanical axis. In fact, these reinforced composite polymer materials can even approach the strength and stiffness of cortical bone, making them the first absorbable materials for use in load bearing orthopedic implant applications.

On an underlying level, there is a great difference between the reinforced biocomposite implants and previous implants from metal, plastic, and other traditional medical implant materials. Traditional medical implant materials are isotropic such that their mechanical properties are identical in all axes. This simplifies implant design as the mechanical strength of the implant is determined solely based on the geometry of the implant and the inherent material properties of the material. Without wishing to be limited by a closed list or a single hypothesis, for reinforced biocomposite implants, the inherent material properties of the biocomposite (i.e. the biocomposite in amorphous or non-aligned form) are actually quite low and can approximate the mechanical properties of the polymer alone. As such, implant geometries for implants constructed from these biocomposite materials does not inherently determine implants that are mechanically strong or stiff.

However, the medical implants of the present invention in at least some embodiments are able to exceed the mechanical properties of previous bioabsorbable implants, including previous biocomposite implants in one or more mechanical axes and in one or more mechanical parameters. Preferably these implants feature structures and forms in which the reinforcing fibers are aligned within the implant in order to provide the implant load bearing strength and stiffness in the axes in which these properties are biomechanically required. Thus, either the entire implant or segments of the implant are anisotropic (i.e. they have different mechanical properties in different axes). With these anisotropic implants, the implant mechanical design cannot rely solely on the geometry of each part. Rather, the specific alignment of the reinforcing fibers within the implant and the resulting anisotropic mechanical profile are a key parameter in determining the biomechanical function of the implant.

Aside from the mechanical considerations related to the anisotropic medical implants, there are additional limitations in that medical implants using these reinforced biocomposite materials cannot be designed according to existing implant designs due to the limitations associated with producing parts from these composite materials.

For example, metal implants or permanent polymer implants may be produced by machining. Even fiber-reinforced permanent polymer implants may be machined without adversely affecting the mechanical properties. However, absorbable, reinforced composite material implants cannot be machined without causing damage to the underlying material since machining will expose reinforcing fibers from the polymer, thus causing their strength to degrade quickly once they are directly exposed to body fluid following implantation.

At the other end of the spectrum, pure polymer or very short (<4 mm) fiber-reinforced polymer implants may be manufactured using straightforward injection molding processes. Injection molding of these materials does not, however, result in sufficiently strong implants. Therefore, specialized designs and production methods are required in order to design and produce an implant that can benefit from the superior mechanical properties of the previously described reinforced bioabsorbable composite materials.

The term “biodegradable” as used herein also refers to materials that are degradable, resorbable or absorbable in the body.

The term “load bearing” optionally also includes partially load bearing. According to various embodiments, the load bearing nature of the device (implant) may optionally include flexural strengths above 200 MPa, preferably above 300 MPa, more preferably above 400 MPa, 500 MPa, and most preferably above 600 MPa or any integral value in between.

The biocomposite orthopedic implants as described herein feature a composite of mineral compositions and bioabsorbable polymer. Optionally, a majority or entirety of the surface of the implant is comprised of the bioabsorbable polymer. This may result from the underlying composition of the biocomposite or from the production method (such as injection or compression molding) used to produce the implant. However, the attachment and incorporation of bone to the mineral composition component of the implant is generally better than the attachment of bone to the polymer. This may be due to one or more factors including the relative hydrophilicity of the mineral component as compared with the polymer component, increased porosity of the mineral component as compared with the polymer component, or additional factors.

Therefore, in order to improve the attachment of bone to a biocomposite implant, according to at least some embodiments of the present invention, there is provided such an implant in which the percentage of the implant surface that is comprised of mineral component is maximized. Optionally and preferably, such maximization is implemented by introducing roughness or porosity to the surface of the biocomposite implant to further improve the bone attachment to the biocomposite implant.

It should be noted that such maximization may optionally be implemented with any biocomposite implant featuring a combination of mineral compositions and bioabsorbable polymer, and not only such implants as described herein.

According to at least some embodiments, there is provided a method of precise ablation of polymer material from a surface of an implant, and implants with such an ablated surface. The depth to which this occurs is preferably controlled. Also preferably the structure of fiber is preserved, such that only the surface polymer is removed. There is preferably a low amount of depth variation and square area variation.

Ablation can optionally be achieved through any suitable method, including but not limited to an erosive method, including mechanical brushing, cutting or chipping, and/or irradiation or laser ablation. Preferably ablation is achieved through irradiation or laser ablation.

The polymer material is ablated from the surface of the implant without damaging the mineral fibers and without compressing them so they retain their structure intact and are not frayed. In terms of fiber diameter, preferably the fiber diameter ranges in a range of 2-40 microns, and more preferably 4-20 microns fiber diameter.

Preferably the polymer surface is ablated to a controlled extent, such that a structure of said fibers is maintained upon ablation of the polymer surface. Also preferably the fiber structure is maintained, wherein at least 50, 65, 80, 85, 90, 95% of surface fibers retain their geometric structure or are not ablated or do not have a portion of the fiber removed. For example, optionally the depth of the polymer surface is in the range of 1-100 micron, 5-50 micron, or 10-30 micron. Optionally the fiber diameter is in a range of 2-40 microns, and more preferably 4-20 microns fiber diameter.

Preferably the polymer surface varies across different cross-sections of the medical implant. Optionally the depth of the polymer surface is in the range of 1-50 microns in one cross-section of the implant and greater than 50 microns in another cross section.

Also optionally the depth of the polymer surface is in the range of 5-50 microns in one cross-section of the implant and greater than 100 microns in another cross section.

Preferably the depth of the polymer surface is not more than 100 microns, 90, 80, 70, 60, 50, 40, 30, 20 or anything in between.

In various embodiments, different amounts of the outer surface of the medical implant may be surface treated with ablation. For example, 10-70% of the medical implant outer surface may be surface treated with ablation. Optionally, 30-55% of the medical implant outer surface is surface treated. Also optionally, 15-40% of the medical implant outer surface is surface treated.

Various embodiments may feature different depths of ablation, as measured from the outer surface of the implant. For example, a depth of the ablation optionally ranges from 1 to 120 microns from outer surface. Optionally the depth of the ablation ranges from 5 to 70 microns. Also optionally, the depth of the ablation ranges from 5 to 40 microns.

In various exemplary implementations of the implant, the fibers are arranged in layers. Optionally, the depth of the ablation ranges from 1 to 50 microns into the top layer of the fibers. Preferably, the depth of the ablation ranges from 3 to 20 microns.

Ablation depth may be considered with regard to implant wall thickness and/or overall implant thickness. For example, the ablation depth may be in a range of 0.1% to 10% of implant wall thickness or implant overall thickness. Optionally, the depth of the ablation ranges from 0.5% to 2.5%.

Optionally a shape of an area of treatment of the outer surface is selected from the group consisting of rectangular, square, circular, arc shaped, diamond, parallelograms, triangular or any combination of said shapes.

Optionally a shape of an area of treatment of the outer surface comprises a line shape of specified width, wherein said surface treated line width is up to 100 microns. For example, optionally the line shape comprises one or more of continuous solid line, dashed line, dotted line, circumferential line, angled line (any angle from 5 to 85 degrees), helix line (helix angle of 5 to 85 degrees). Preferably the width is in a range of from 5 microns to 100. Optionally the surface treated line width is in a range of from 10 microns to 70 microns. Preferably the surface treated line width is in a range of from 20 microns to 40 microns.

According to various embodiments, the fibers may have different orientations in the implant. For example, optionally the surface treatment exposes fibers of different orientations on the outer surface. Optionally the exposed fiber orientation is parallel to the medical implant body axis. Also optionally, the exposed fiber orientation is 5°-85° relative to the implant body axis. Preferably, the exposed fiber orientation is 15°-65° relative to the implant body axis. More preferably, the exposed fiber orientation is 30°-60° relative to the implant body axis.

Optionally, the exposed fibers have more than one direction on the treated surface. These different directions may be realized, for example, according to an angle between neighboring areas having different fiber directions. Optionally, the angle between one area of fiber direction to its neighboring area with different fiber direction is between 0°-90°. Also optionally, the angle between one area of fiber direction to its neighboring area with different fiber direction is between 25°-75°. Optionally, the cross-sectional fiber exposure is 90° relative to the fiber axis. Optionally, the cross-sectional fiber exposure is 15°-65° relative to the fiber axis. Optionally, the cross-sectional fiber exposure comprises more than one fiber direction.

Surface maximum roughness may also be controlled through ablation. Controlling surface maximum roughness may, without wishing to be limited by a closed list, lead to such benefits as better ingrowth of tissue, better adherence to tissue and so forth. Optionally surface maximum roughness is more than 1-10 microns. Preferably the surface maximum roughness is more than 3-8 microns. Also preferably, the surface maximum roughness is more than 4-6 microns.

After ablation, various resultant surface geometries may occur in regard to the exposed fibers. For example, the surface may be exposed fibers alone. The exposed fibers may comprise 20-80% of the ablated surface. Optionally, the exposed fibers comprise 35-65% of the ablated surface. Optionally, the exposed fibers comprise 51-70% of the ablated surface.

After ablation, various surface geometries may also be provided with different shapes. For example, optionally after ablation the resultant surface geometry is step shaped.

Optionally the implant may comprise a plurality of ribs or threads, wherein said polymer surface is thicker on the implant body comparing to said polymer surface thickness on the ribs/threads. For example, the ribs/threads may be treated while a remainder of the implant is untreated, or vice versa.

Unless otherwise defined, all technical and scientific terms used herein have the same meaning as commonly understood by one of ordinary skill in the art to which this invention belongs. The materials, methods, and examples provided herein are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 shows surface texture as imaged by an SEM representing the surface of the implant as it comes out of the mold (A) and after surface treatment (B). Surface roughness is increased, and small nm (nanometer) and micron holes can be seen due to the treatment, which facilitate cell in-growth and degradation.

FIG. 2 shows surface texture as imaged by an SEM representing the surface of the implant as it comes out of the mold (A) and after surface treatment (B). Surface roughness is increased. Image was taken at a different magnification.

FIG. 3 shows implant mineral fibers partially exposed as imaged by an SEM before treatment (A) and after surface treatment (B). Fiber exposure increased due to the surface treatment.

FIG. 4 shows surface texture as imaged by an SEM representing the surface of the implant as it comes out of the mold (A) and after surface treatment (B). Surface roughness is increased, ˜200 micron holes can be seen due to the treatment, which facilitate cell in-growth and degradation.

FIG. 5 shows surface cross-section as imaged by a scanning electron microscope (SEM) (FEI Quanta FEG 250, Holland) showing representative measurements of an implant outer surface layer, in this case 17.6±6.8 micron. Reference numbers: 101, 107, 113, 119 represent fibers, 103, 109, 111, 115 represent the polymer edge, 105 represents the measurement from the edge to the closest fiber.

FIGS. 6A and 6B show surface texture as imaged by a scanning electron microscope (SEM) (FEI Quanta FEG 250, Holland) representing the surface of the implant as it comes out of the mold (A) and after surface treatment (B). Surface roughness is increased, small nm and micron holes can be seen due to the treatment, which facilitate cell in-growth and degradation.

FIGS. 7A and 7B show images by a Focused ion beam setup (FIB) (Helios 600, FEI) representing (A) the surface of the implant after treatment which creates ˜1 micron features and (B) a cross-section cut made by the FIB showing representative dimensions of a 45 micron surface layer which has a different composition than the inner material. Surface includes in this case a combination of a ˜2.5 micron outer thick layer were the roughness is increased, small nm and micron holes can be seen due to the treatment, which facilitate initial cell attachment and ˜40 micron layer of polymer. The cross section of the two mineral fibers can also be seen in the image.

FIG. 8 shows an implant cross-section imaged by a scanning electron microscope (SEM) (FEI Quanta FEG 250, Holland) representing an implant where less than 60% of the circumference, which represents the less than 60% of the surface area is different in composition than the inner composition of the implant.

FIG. 9 shows surface texture as imaged by a scanning electron microscope (SEM) (FEI Quanta FEG 250, Holland) representing the surface of the implant after CNC machining treatment which partially exposes fiber bundles (white arrows). Exposed fibers can be seen due to the treatment, which facilitate cell in-growth and degradation.

FIG. 10 shows an image demonstrating continuous fiber body composition.

FIG. 11 shows schematics of a representative implant cross-section. Schematics are not in scale, but include the implant body comprised of one composition, 705, the surface layer which in this case includes both an inner surface layer 703 and an outer surface layer 701, each with a different composition.

FIG. 12 shows biocomposite medical implant with a cross-section view of the layer surface.

FIG. 13 shows biocomposite medical implant with a cross-section view of the layer surface.

FIG. 14 shows biocomposite medical implant with a top view of untreated vs. treated surface.

FIG. 15 shows biocomposite medical implant with a top view; ×200 magnification of the treated surface section.

FIG. 16 shows biocomposite medical implant; side view; surface treatment location.

FIG. 17 shows biocomposite medical implant; side view; directional fiber orientation exposure.

FIG. 18 shows biocomposite medical implant; side view; directional fiber orientation exposure with magnification on the treated surface border line.

FIG. 19 shows biocomposite medical implant; side view; two different fiber orientations on the same surface.

FIG. 20 shows biocomposite medical implant; side view; two different fiber orientations on the same surface.

FIG. 21 shows biocomposite medical implant; cross-section view perpendicular to fiber axis.

FIG. 22 shows biocomposite medical implant; cross-section view ˜45° relative to fiber axis.

FIG. 23 shows biocomposite medical implant; cross-section view ˜10° relative to fiber axis.

FIGS. 24A-D show biocomposite medical implant; different surface roughness and geometries as obtained by four different surface treatment methods.

FIG. 25 shows biocomposite medical implant; top view; different outer surface composition as a result of different surface treatments.

FIG. 26 shows a hexagonal ribbed pin implant; side view and front view.

FIG. 27 shows a front view of implant position for laser ablation.

FIG. 28 shows an ablated surface illustration; ablation surfaces are marked in black.

FIG. 29 shows a laser focal point line position on the ablated hex face.

FIG. 30 shows a hexagonal ribbed pin implant before wafer removal; side view and front view.

FIG. 31 shows a hexagonal ribbed pin implant after wafer removal; side view and front view.

FIG. 32 shows a side view of implant position for laser fiber cross-section exposure.

FIG. 33 shows an ablated surfaces illustration; ablation surfaces are marked in black.

FIG. 34 shows laser focal point line position on the ablated hex face.

### DETAILED DESCRIPTION OF SOME EMBODIMENTS

A medical implant according to at least some embodiments of the present invention is suitable for load-bearing orthopedic implant applications and comprises one or more bioabsorbable materials where sustained mechanical strength and stiffness are critical for proper implant function.

According to at least some embodiments of the present invention, there is provided orthopedic implants, such as those for bone fixation, made from reinforced bioabsorbable composite materials. Specifically, implants according to at least some embodiments incorporate characteristics, features, or properties that can either only be achieved using the reinforced bioabsorbable composite materials or are specifically advantageous for implants comprised of these types of materials, or optionally a combination of both in a single implant.

Surface and Body Compositions

According to at least some embodiments, the reinforced biocomposite medical implant is comprised of an internal composition region, or “body,” and a surface region, defined as the region comprising the surface layer of part or all of the implant.

The surface region may be further broken down into an outermost (external) surface region and innermost (internal) surface region, each of which may have different properties.

The surface region may cover the entire surface of the implant but can also cover only a percentage of the surface of the implant, with the remaining surface being of the same properties as the internal composition region. Preferably, surface region covers at least a majority of the entire surface of the implant.

Optionally, one or more cannulation or screw hole voids may be present on the inside of implant, which may or may not be included in the calculation of implant surface.

Surface region can be defined as a layer of average depth in the range of 0.1-200 micron, preferably 1-100 micron, more preferably 2-75 micron and most preferably 5-50 micron.

Outermost surface region can be defined as the external layer of the surface region with an average depth in the range of 0.1-100 micron, preferably 0.5-50 micron, more preferably 1-25 micron and most preferably 1-10 micron.

In one embodiment, the implant is a mineral fiber-reinforced biocomposite implant and fewer reinforcing fibers are present in either the entire surface region or the outermost surface region as compared with the internal composition region. Preferably, fiber to polymer weight composition ration in surface region is less than 50% of fiber to polymer weight ratio in internal composition region. More preferably less than 30%, and most preferably less than 10%. Optionally, no fibers are present in surface region or the outermost surface region.

In one embodiment, outermost surface region has been modified to increase roughness and/or porosity.

Optionally roughness is defined by presence of promontories, prominences or protuberances on the surface of the implant with height equal to or less than the depth of the outermost surface region. Preferably such promontories, prominences or protuberances are less than 5 microns in diameter, on average. More preferably, less than 3, less than 2, less than 1 micron in average diameter. Optionally such promontories, prominences or protuberances are present in the outermost surface area but absent in the innermost surface area.

Optionally roughness is defined by Ra measure in nanometers (nm). Preferably roughness in modified outermost surface area is greater than 100 nm, more preferably greater than 200 nm, and most preferably greater than 300 nm. Preferably roughness in unmodified surface area is less than 100 nm.

Optionally, porosity is defined as full thickness pore (holes) in the entire surface region or outermost surface layer. Preferably, implant is a mineral fiber-reinforced implant and porosity in surface layer exposes mineral fibers.

Optionally, the surface region has lower mineral content than the internal composition region.

Optionally, the internal composition region has:

Sodium (Na) weight composition of 1-10%, preferably 2-8%, and more preferably 3-6%.

Magnesium (Mg) weight composition of 0.4-1.5%, preferably 0.4-1.2%, and more preferably 0.8-1.2%.

Silica (Si) weight composition of 1-20%, preferably 5-15%, and more preferably 9-13%.

Phosphorous (P) weight composition of less than 3%, preferably less than 1%.

Calcium (Ca) weight composition of 1-20%, preferably 1-10%, preferably 1-3%.

Optionally, the innermost surface region has lower mineral content than internal composition region.

Optionally, the innermost surface region has:

Sodium (Na) weight composition of less than 1.9%, preferably less than 1.5%. Preferably the sodium weight composition of innermost surface region is 50% less than sodium weight composition of internal composition and more preferably 30% less.

Magnesium (Mg) weight composition of less than 0.3%, preferably less than 0.2%. Preferably the magnesium weight composition of innermost surface region is 50% less than magnesium weight composition of internal composition and more preferably 30% less.

Silica (Si) weight composition of less than 6%, preferably less than 4%. Preferably silica weight composition of innermost surface region is 50% less than silica weight composition of internal composition and more preferably 30% less.

Phosphorous (P) weight composition of less than 3%, preferably less than 1%.

Calcium (Ca) weight composition of less than 1%, preferably less than 0.5%. Preferably calcium weight composition of innermost surface region is 50% less than calcium weight composition of internal composition and more preferably 30% less.

Optionally, the outermost surface region has higher mineral content than the innermost surface region.

Optionally, outermost surface region has:

Sodium (Na) weight composition of less than 1.9%, preferably less than 1.5%.

Magnesium (Mg) weight composition of less than 1%, preferably less than 0.5%. Preferably magnesium weight composition of outermost surface region is greater than magnesium weight composition of innermost surface region.

Silica (Si) weight composition of less than 6%, preferably less than 4%. Preferably silica weight composition of outermost surface region is 50% less than silica weight composition of internal composition and more preferably 30% less.

Phosphorous (P) weight composition in range of 1-15%, preferably 3-13%. Preferably phosphorous weight composition of outermost surface region is at least 50% greater than phosphorous weight composition of innermost layer or than internal composition or than both; more preferably at least 70% greater and most preferably at least 90% greater.

Calcium (Ca) weight composition in range of 15-50%, preferably 15-30%. Preferably calcium weight composition of outermost surface region is at least 100% greater than calcium weight composition of innermost layer, more preferably at least 500% greater and most preferably at least 1000% greater.

Biocomposite Implants with Modified Surface Area

According to at least some embodiments, there is provided a biocomposite medical implant with a modified surface wherein the outermost surface layer of the implant is comprised of a majority of bioabsorbable polymer but wherein the surface has been modified such that the surface of the implant comprises roughness, texture, or porosity such that an increased amount of mineral composition is exposed as compared with the outermost surface layer of the implant.

Outermost surface layer as used herein may define the outermost 1-100 μm of the implant. Preferably the outermost 1-20 μm of the implant, more preferable the outermost 1-10, and most preferably the outer 1-5.

The exposed mineral composition may comprise the mineral composition that is part of the biocomposite composition. The mineral composition may optionally or additionally comprise another mineral such as Hydroxyapatite, Calcium Phosphate, Calcium Sulfate, Dicalcium Phosphate, Tricalcium Phosphate.

The roughness or texture of the surface may include exposure of the internal composition of the implant to a depth of the outermost 1-100 μm of the implant. Preferably the outermost 1-20 μm of the implant, more preferable the outermost 1-10, and most preferably the outer 1-5 microns.

Preferably, the outermost layer of the implant comprises at least 30% polymer, more preferably at least 50%, more preferably at least 70%, and most preferably at least 80%.

The composition of the biocomposite is comprised of at least 20% mineral composition, preferably at least 30%, more preferably at least 40%, and most preferably at least 50%.

Preferably the composition of the outermost layer of the implant comprises a greater percentage of polymer than the overall composition of the implant. Preferably, at least 10% more, 20%, 30%, 50%

Optionally, the modified surface of the implant includes pores in the polymer surface. The average pore diameter is preferably in the range of 1-500 μm, more preferably in the range 10-300 μm, more preferably in the range 50-250 μm.

Preferably, surface is modified with surface treatment using grit blasting.

Preferably grit is comprised of a biocompatible material.

Preferably grit is comprised of a combination of Hydroxyapatite, Calcium Phosphate, Calcium Sulfate, Dicalcium Phosphate, and Tricalcium Phosphate.

Preferably grit is of an average diameter size in the range of 10-500 μm. More preferably in the range of 20-120 μm.

Bioabsorbable Polymers

In a preferred embodiment of the present invention, the biodegradable composite comprises a bioabsorbable polymer.

The medical implant described herein may be made from any biodegradable polymer. The biodegradable polymer may be a homopolymer or a copolymer, including random copolymer, block copolymer, or graft copolymer. The biodegradable polymer may be a linear polymer, a branched polymer, or a dendrimer. The biodegradable polymers may be of natural or synthetic origin. Examples of suitable biodegradable polymers include, but are not limited to polymers such as those made from lactide, glycolide, caprolactone, valerolactone, carbonates (e.g., trimethylene carbonate, tetramethylene carbonate, and the like), dioxanones (e.g., 1,4-dioxanone), δ-valerolactone, 1,dioxepanones) e.g., 1,4-dioxepan-2-one and 1,5-dioxepan-2-one), ethylene glycol, ethylene oxide, esteramides, γ-ydroxyvalerate, β-hydroxypropionate, alpha-hydroxy acid, hydroxybuterates, poly (ortho esters), hydroxy alkanoates, tyrosine carbonates, polyimide carbonates, polyimino carbonates such as poly (bisphenol A-iminocarbonate) and poly (hydroquinone-iminocarbonate, (polyurethanes, polyanhydrides, polymer drugs (e.g., polydiflunisol, polyaspirin, and protein therapeutics (and copolymers and combinations thereof. Suitable natural biodegradable polymers include those made from collagen, chitin, chitosan, cellulose, poly (amino acids), polysaccharides, hyaluronic acid, gut, copolymers and derivatives and combinations thereof.

According to the present invention, the biodegradable polymer may be a copolymer or terpolymer, for example: polylactides (PLA), poly-L-lactide (PLLA), poly-DL-lactide (PDLLA); polyglycolide (PGA); copolymers of glycolide, glycolide/trimethylene carbonate copolymers (PGA/TMC); other copolymers of PLA, such as lactide/tetramethylglycolide copolymers, lactide/trimethylene carbonate copolymers, lactide/d-valerolactone copolymers, lactide/ε-caprolactone copolymers, L-lactide/DL-lactide copolymers, glycolide/L-lactide copolymers (PGA/PLLA), polylactide-co-glycolide; terpolymers of PLA, such as lactide/glycolide/trimethylene carbonate terpolymers, lactide/glycolide/ε-caprolactone terpolymers, PLA/polyethylene oxide copolymers; polydepsipeptides; unsymmetrically—3,6-substituted poly-1,4-dioxane-2,5-diones; polyhydroxyalkanoates; such as polyhydroxybutyrates (PHB); PHB/b-hydroxyvalerate copolymers (PHB/PHV); poly-b-hydroxypropionate (PHPA); poly-p-dioxanone (PDS); poly-d-valerolactone-poly-ε-capralactone, poly(ε-caprolactone-DL-lactide) copolymers; methylmethacrylate-N-vinyl pyrrolidone copolymers; polyesteramides; polyesters of oxalic acid; polydihydropyrans; polyalkyl-2-cyanoacrylates; polyurethanes (PU); polyvinylalcohol (PVA); polypeptides; poly-b-malic acid (PMLA): poly-b-alkanbic acids; polycarbonates; polyorthoesters; polyphosphates; poly(ester anhydrides); and mixtures thereof; and natural polymers, such as sugars; starch, cellulose and cellulose derivatives, polysaccharides, collagen, chitosan, fibrin, hyalyronic acid, polypeptides and proteins. Mixtures of any of the above-mentioned polymers and their various forms may also be used.

The biodegradable composite is preferably embodied in a polymer matrix, which may optionally comprise any of the above polymers. Optionally and preferably, it may comprise a polymer selected from the group consisting of a bioabsorbable polyester, PLLA (poly-L-lactide), PDLLA (poly-DL-lactide), PLDLA, PGA (poly-glycolic acid), PLGA (poly-lactide-glycolic acid), PCL (Polycaprolactone), PLLA-PCL and a combination thereof. If PLLA is used, the matrix preferably comprises at least 30% PLLA, more preferably 50%, and most preferably at least 70% PLLA. If PDLA is used, the matrix preferably comprises at least 5% PDLA, more preferably at least 10%, most preferably at least 20% PDLA.

Optionally, the inherent viscosity (IV) of the polymer matrix (independent of the reinforcement fiber) is in the range of 0.2-6 dl/g, preferably 1.0 to 3.0 dl/g, more preferably in the range of 1.5 to 2.4 dl/g, and most preferably in the range of 1.6 to 2.0 dl/g.

Inherent Viscosity (IV) is a viscometric method for measuring molecular size. IV is based on the flow time of a polymer solution through a narrow capillary relative to the flow time of the pure solvent through the capillary.

Reinforced Biocomposite

According to at least some embodiments of the present invention, the medical implant comprises a reinforced biocomposite (i.e. a bioabsorbable composite that includes the previously described polymer and also incorporates a reinforcing filler, generally in fiber form, to increase the mechanical strength of the polymer). For the avoidance of doubt, the terms “filler” and “fiber” are used interchangeably to describe the reinforcing material structure.

In a more preferred embodiment of the present invention, the reinforced bioabsorbable polymer is a reinforced polymer composition comprised of any of the above-mentioned bioabsorbable polymers and a reinforcing filler, preferably in fiber form. The reinforcing filler may be comprised of organic or inorganic (that is, natural or synthetic) material. Reinforcing filler may be a biodegradable glass or glass-like materials, a ceramic, a mineral composition (optionally including one or more of hydroxyapatite, tricalcium phosphate, calcium sulfate, calcium phosphate), a cellulosic material, a nano-diamond, or any other filler known in the art to increase the mechanical properties of a bioabsorbable polymer. The filler may also optionally be a fiber of a bioabsorbable polymer itself. Preferably, reinforcing fiber is comprised of a bioabsorbable glass, ceramic, or mineral composition.

Preferably, reinforcement fiber is comprised of silica-based mineral compound such that reinforcement fiber comprises a bioresorbable glass fiber, which can also be termed a bioglass fiber composite.

According to at least some embodiments, bioresorbable glass fiber may optionally have oxide compositions in the following mol. % ranges (as a percent over the glass fiber composition):

### Na₂O: 11.0-19.0 mol. %

### CaO: 9.0-14.0 mol. %

### MgO: 1.5-8.0 mol. %

B₂O₃: 0.5-3.0 mol. %
Al₂O₃: 0-0.8 mol. %
P₂O₃: 0.1-0.8 mol. %

### SiO₂: 67-73 mol. %

but preferably in the following mol. % ranges:

### Na₂O: 12.0-13.0 mol. %

### CaO: 9.0-10.0 mol. %

### MgO: 7.0-8.0 mol. %

B₂O₃: 1.4-2.0 mol. %
P₂O₃: 0.5-0.8 mol. %

### SiO₂: 68-70 mol. %

Additional optional bioresorbable glass compositions are described in the following patent applications, which are hereby incorporated by reference as if fully set forth herein: Biocompatible composite and its use (WO2010122098); and Resorbable and biocompatible fibre glass compositions and their uses (WO2010122019).

Tensile strength of the reinforcement fiber is preferably in the range of 1200-2800 MPa, more preferably in the range of 1600-2400 MPa, and most preferably in the range of 1800-2200 MPa.

Elastic modulus of the reinforcement fiber is preferably in the range of 30-100 GPa, more preferably in the range of 50-80 GPa, and most preferably in the range of 60-70 GPa.

Reinforcing filler is preferably incorporated in the bioabsorbable polymer matrix of the biocomposite in fiber form. Preferably, such fibers are continuous fibers.

Preferably continuous fibers are aligned within the implant such that the ends of fibers don't open at the surface of the implant.

Preferably, fibers are distributed evenly within the implant.

Specifically within bioabsorbable fiber-reinforced composites, achieving the high strengths and stiffness required for many medical implant applications can require the use of continuous-fiber reinforcement rather than short or long fiber reinforcement. This creates a significant difference from the implant structures, architectures, designs, and production techniques that have been previously used with medical implants produced from polymers or composites comprising short or long fiber reinforced polymers. Those implants are most commonly produced using injection molding, or occasionally 3-D printing, production techniques. The production of these implants generally involves homogeneity of the material throughout the implant and the finished implant is then comprised of predominantly isotropic material. However, with continuous fiber-reinforcement, the fibers must be carefully aligned such that each fiber or bundle of fibers runs along a path within the composite material such that they will provide reinforcement along specific axes within the implant to provide stress resistance where it is most needed.

The present invention provides, in at least some embodiments, implant compositions from continuous-fiber reinforced bioabsorbable composite materials that are a significant step forward from previous bioabsorbable implants in that they can achieve sustainably high, load bearing strengths and stiffness. Additionally, many embodiments of the present invention additionally facilitate these high strength levels with efficient implants of low volume since the anisotropic nature of the implants can allow the implants to achieve high mechanical properties in axes where those properties are needed (for example in bending resistance) without necessitating the additional volume that would be needed to uniformly provide high mechanical properties in all other axes.

According to at least some embodiments, there is provided a medical implant comprising a plurality of composite layers, said layers comprising a biodegradable polymer and a plurality of uni-directionally aligned continuous reinforcement fibers. Optionally and preferably, the biodegradable polymer is embodied in a biodegradable composite. Also optionally and preferably, the fibers are embedded in a polymer matrix comprising one or more bioabsorbable polymers.

According to at least some embodiments, the composite layers are each comprised of one or more composite tapes, said tape comprising a biodegradable polymer and a plurality of uni-directionally aligned continuous reinforcement fibers. Optionally and preferably, the biodegradable polymer is embodied in a biodegradable composite. Also optionally and preferably, the fibers are embedded in a polymer matrix comprising one or more bioabsorbable polymers.

Preferably, the composite tape layer comprises reinforcement fibers that are pre-impregnated with polymer.

Preferably, each composite layer is of thickness 0.05 mm-0.5 mm, more preferably 0.15-0.35 mm, and most preferably 0.1-0.25 mm.

Preferably, each composite tape is of width 2-30 mm, more preferably tape is of width 4-16 mm, and most preferably of width 6-12 mm.

Preferably, reinforcement fiber content within the composite tape is in the range of 20-70%, more preferably in the range of 30-60%, more preferably in the range of 40-50%, and most preferably 45-50% over the entire composite tape materials.

Optionally and preferably, the fiber-reinforced biodegradable composite within the implant has a flexural modulus exceeding 10 GPa and flexural strength exceeding 100 MPa.

Optionally, the fiber-reinforced biodegradable composite within the implant has flexural strength in range of 200-1000 MPa, preferably 300-800 MPa, more preferably in the range of 400-800 MPa, and most preferably in the range of 500-800 MPa

Optionally, the fiber-reinforced biodegradable composite within the implant has elastic modulus in range of 10-30 GPa, preferably 12-28 GPa, more preferably in the range of 16-28 GPa, and most preferably in the range of 20-26 GPa.

Optionally, fibers may be aligned at an angle to the longitudinal axis (i.e. on a diagonal) such that the length of the fiber may be greater than 100% of the length of the implant. Optionally and preferably, a majority of reinforcement fibers are aligned at an angle that is less than 90°, alternatively less than 60°, or optionally less than 45° from the longitudinal axis.

Preferably, the implant preferably comprises between 2-20 composite tape layers, more preferably between 2-10 layers, and most preferably between 2-6 layers; wherein each layer may be aligned in a different direction or some of the layers may be aligned in the same direction as the other layers.

Preferably, the maximum angle between fibers in at least some of the layers is greater than the angle between the fibers in each layer and the longitudinal axis. For example, one layer of reinforcing fibers may be aligned and a right diagonal to the longitudinal axis while another layer may be aligned at a left diagonal to the longitudinal axis.

Optionally and preferably, the composite composition additionally includes a compatibilizer, which for example be such an agent as described in WO2010122098, hereby incorporated by reference as if fully set forth herein.

Reinforcing fiber diameter preferably in range of 2-40 um, preferably 8-20 um, most preferably 12-18 um (microns).

Preferably, the implant includes only one composition of reinforcing fiber.

Preferably fibers don't open at the surface of the implant.

Numerous examples of reinforced polymer compositions have previously been documented. For example: A biocompatible and resorbable melt derived glass composition where glass fibers can be embedded in a continuous polymer matrix (EP 2 243 749 A1), Biodegradable composite comprising a biodegradable polymer and 20-70 vol % glass fibers (WO2010128039 A1), Resorbable and biocompatible fiber glass that can be embedded in polymer matrix (US 2012/0040002 A1), Biocompatible composite and its use (US 2012/0040015 A1), Absorbable polymer containing poly[succinimide] as a filler (EP0 671 177 B1).

In a more preferred embodiment of the present invention, the reinforcing filler is covalently bound to the bioabsorbable polymer such that the reinforcing effect is maintained for an extended period. Such an approach has been described in US 2012/0040002 A1 and EP 2243500B1, hereby incorporated by reference as if fully forth herein, which discusses a composite material comprising biocompatible glass, a biocompatible matrix polymer and a coupling agent capable of forming covalent bonds.

Fabrication of the Implant

Any of the above-described bioabsorbable polymers or reinforced bioabsorbable polymers may be fabricated into any desired physical form for use with the present invention. The polymeric substrate may be fabricated for example, by compression molding, casting, injection molding, pultrusion, extrusion, filament winding, composite flow molding (CFM), machining, or any other fabrication technique known to those skilled in the art. The polymer may be made into any shape, such as, for example, a plate, screw, nail, fiber, sheet, rod, staple, clip, needle, tube, foam, or any other configuration suitable for a medical device.

Load-Bearing Mechanical Strength

The present invention particularly relates to bioabsorbable composite materials that can be used in medical applications that require high strength and a stiffness compared to the stiffness of bone. These medical applications require the medical implant to bear all or part of the load applied by or to the body and can therefore be referred to generally as “load-bearing” applications. These include bone fixation, fracture fixation, tendon reattachment, joint replacement, spinal fixation, and spinal cages.

The flexural strength preferred from a bioabsorbable composite (such as a reinforced bioabsorbable polymer) for use in the load-bearing medical implant is at least 200 MPa, preferably above 400 MPa, more preferably above 600 MPa, and even more preferably above 800 MPa. The Elastic Modulus (or Young's Modulus) of the bioabsorbable composite for use with present invention is preferably at least 10 GPa, more preferably above 15 GPa, and even more preferably above 20 GPa but not exceeding 100 GPa and preferably not exceeding 60 GPa.

Sustained Mechanical Strength

There is a need for the bioabsorbable load-bearing medical implants of the present invention to maintain their mechanical properties (high strength and stiffness) for an extended period to allow for sufficient bone healing. The strength and stiffness preferably remains above the strength and stiffness of cortical bone, approximately 150-250 MPa and 15-25 GPa respectively, for a period of at least 3 months, preferably at least 6 months, and even more preferably for at least 9 months in vivo (i.e. in a physiological environment).

More preferably, the flexural strength remains above 400 MPa and even more preferably remains above 600 MPa.

The present invention overcomes the limitations of previous approaches and provides medical implants comprised of biodegradable compositions that retain their high mechanical strength and stiffness for an extended period sufficient to fully support bone regeneration and rehabilitation.

“Biodegradable” as used herein is a generalized term that includes materials, for example polymers, which break down due to degradation with dispersion in vivo. The decrease in mass of the biodegradable material within the body may be the result of a passive process, which is catalyzed by the physicochemical conditions (e.g. humidity, pH value) within the host tissue. In a preferred embodiment of biodegradable, the decrease in mass of the biodegradable material within the body may also be eliminated through natural pathways either because of simple filtration of degradation by-products or after the material's metabolism (“Bioresorption” or “Bioabsorption”). In either case, the decrease in mass may result in a partial or total elimination of the initial foreign material. In a preferred embodiment, said biodegradable composite comprises a biodegradable polymer that undergoes a chain cleavage due to macromolecular degradation in an aqueous environment.

A polymer is “absorbable” as described herein if it is capable of breaking down into small, non-toxic segments which can be metabolized or eliminated from the body without harm. Generally, absorbable polymers swell, hydrolyze, and degrade upon exposure to bodily tissue, resulting in a significant weight loss. The hydrolysis reaction may be enzymatically catalyzed in some cases. Complete bioabsorption, i.e. complete weight loss, may take some time, although preferably complete bioabsorption occurs within 24 months, most preferably within 12 months.

The term “polymer degradation” means a decrease in the molecular weight of the respective polymer. With respect to the polymers, which are preferably used within the scope of the present invention said degradation is induced by free water due to the cleavage of ester bonds. The degradation of the polymers as for example used in the biomaterial as described in the examples follows the principle of bulk erosion. Thereby a continuous decrease in molecular weight precedes a highly pronounced mass loss. Such loss of mass is attributed to the solubility of the degradation products. Methods for determination of water induced polymer degradation are well known in the art such as titration of the degradation products, viscometry, differential scanning calorimetry (DSC).

Bulk degradation refers to a process of degradation in which there is at least some perfusion of fluid through the material that is being degraded, such as the body of the implant, thereby potentially degrading the bulk of the material of the implant (as opposed to the external surface alone). This process has many effects. Without wishing to be limited to a closed list, such bulk degradation means that simply making an implant larger or thicker may not result in improved retained strength.

Surface degradation refers to a process of degradation in which the external surface undergoes degradation. However, if there is little or no perfusion of fluid through the material that is being degraded, then the portion of the implant that is not on the surface is expected to have improved retained strength over implants in which such perfusion occurs or occurs more extensively.

Material Specific Design Benefits

Without wishing to be limited by a closed list, the material-specific design benefits are optionally provided by one or more of the following unique characteristics of implants manufactured from this material:

1. Absorbable structural implants wherein strength and stiffness properties are anisotropic. The bending resistance and other mechanical properties of these implants depends greatly on the specific design of the part and of the alignment of reinforcing fibers within the part. It is therefore possible to design such implants efficiently such that they provide sufficient support in the necessary axes (for example, flexural stiffness) without comprising an excessive amount of material that would provide equivalent support in the remaining axes (for example, tensile stiffness).

2. Low profile/minimally invasive/material efficient design for absorbable implant that take advantage of the strength and stiffness characteristics of the reinforced absorbable composite material to create implants that achieve bone fixation with minimal profile. By “minimal profile”, it is meant that the implant is reduced in size in at least one dimension in comparison with an equivalent currently available implant that is not made from such composite material.

3. Load bearing absorbable bone implants, as opposed to previous absorbable implants which did not approach the stiffness of cortical bone.

4. Small functional features, such as anchors, ridges, teeth, etc that require the reinforcement in order to be strong enough to be functional. Previous absorbable materials may not have had sufficient strength for such features.

5. The capability of being produced according to fiber-reinforced composite specific manufacturing techniques such as compression molding, pultrusion, etc.

6. Reduced damage to surrounding tissues, including both soft tissues and bone tissues, as compared with the trauma of stress risers or stress shielding that can arise from use of high modulus (such as metal) implants.

The present invention, according to at least some embodiments, thus provides medical implants that are useful as structural fixation for load-bearing purposes, exhibiting sustained mechanical properties.

The present invention, according to at least some embodiments, further comprises a biodegradable composite material in which the drawbacks of the prior art materials can be minimized or even eliminated, i.e. the composite retains its strength and modulus in vivo for a time period sufficient for bone healing for example.

Mechanical strength as used here includes, but is not limited to, bending strength, torsion strength, impact strength, compressive strength and tensile strength.

The presently claimed invention, in at least some embodiments, relate to a biocomposite material comprising a biocompatible polymer and a plurality of reinforcing fibers, wherein said reinforcing fibers are oriented in a parallel orientation.

The biocomposite material has one or more mechanical properties which feature an increased extent or degree as compared to such a material with reinforcing fibers oriented in a non-parallel orientation. Optionally such a non-parallel orientation is a perpendicular or amorphous (non-oriented) orientation. elastic modulus, tensile modulus, compression modulus, shear modulus, bending moment, moment of inertia, bending strength, torsion strength, shear strength, impact strength, compressive strength and/or tensile strength. The increased extent or degree may optionally be at least twice as great, at least five times as great, at least ten times as great, at least twenty times as great, at least fifty times as great, or at least a hundred times as much, or any integral value in between.

Optionally the mechanical properties can comprise any one of Flexural strength, Elastic modulus and Maximum load, any pair of same or all of them. Optionally density and/or volume are unchanged or are similar within 5%, within 10%, within 15%, within 20%, any integral value in between or any integral value up to 50%.

Optionally the biocomposite implant as described herein is swellable, having at least 0.5% swellability, at least 1%, 2% swellability, and less than 20% swellability, preferably less than 10% or any integral value in between.

Optionally, the swellability in one mechanical axis is greater than the swellability in a second mechanical axis. Preferably the difference in swelling percentage (%) between axes is at least 10%, at least 25%, at least 50%, or at least 100%, or any integral value in between.

After exposure to biological conditions for 1 hour, 12 hours, 24 hours, 48 hours, five days, one week, one month, two months or six months or any time value in between, the biocomposite material implants preferably retain at least 10%, at least 20%, at least 50%, at least 60%, at least 75%, at least 85% or up to 100% of flexural strength, Modulus and/or Max load, and/or volume, or any integral value in between. By “biological conditions” it is meant that the temperature is between 30-40 C but preferably is at 37 C. Optionally, fluid conditions replicate those in the body as well, under “simulated body fluid” conditions.

The flexural strength of the implant or segment of the implant is preferably at least 200 MPA, at least 400 mPa, at least 600 mPA, at least 1000 mPA or any integral value in between.

Relevant implants may include bone fixation plates, intramedullary nails, joint (hip, knee, elbow) implants, spine implants, and other devices for such applications such as for fracture fixation, tendon reattachment, spinal fixation, and spinal cages.

According to at least some embodiments, there are provided medical implants for bone or soft tissue fixation comprising a biodegradable composite, wherein said composite optionally and preferably has the following properties:

(i) wherein biodegradable composite comprises one or more biodegradable polymers and a resorbable, reinforcement fiber; and
(ii) wherein one or more segments comprising the medical implant have a maximum flexural modulus in the range of 6 GPa to 30 GPa and flexural strength in the range of 100 MPa to 1000 MPa; and
(iii) wherein the average density of the composite is in the range of 1.1-3.0 g/cm³.

Preferably, average density of the composite is in the range of 1.2-2.0 g/cm³.

More preferably, average density of the composite is in the range of 1.3-1.6 g/cm³.

Preferably, flexural modulus is in the range of 10 GPa to 28 GPa and more preferably in the range of 15 to 25 GPa.

Preferably, flexural strength is in the range of 200-800 MPa. More preferably, 400-800 MPa.

In a preferred embodiment of the present invention, at least 50% of elastic modulus is retained following exposure to simulated body fluid (SBF) at 50° C. for 3 days. More preferably at least 70% is retained, and even more preferably at least 80% is retained.

In a preferred embodiment of the present invention, at least 20% of strength is retained following exposure to simulated body fluid (SBF) at 50° C. for 3 days. More preferably at least 30% is retained, and even more preferably at least 40% is retained.

In a preferred embodiment of the present invention, at least 50% of elastic modulus is retained following exposure to simulated body fluid (SBF) at 37° C. for 3 days. More preferably at least 70%, and even more preferably at least 85%.

In a preferred embodiment of the present invention, at least 30% of strength is retained following exposure to simulated body fluid (SBF) at 37° C. for 3 days. More preferably at least 45%, and even more preferably at least 60%.

Specifically regarding medical implants described herein that contain one or more segments that can be anisotropic, this anisotropicity reflects a significant divergence from what has be previously accepted in medical, and specifically orthopedic, implants in that the anisotropic structure results in implants in which there are mechanical properties in one or more axis that are less than the optimal mechanical properties which may be achieved by the materials from which the implant is comprised. In contrast, traditional implants have relied upon the uniform mechanical properties of the materials from which they are comprised as this does not require compromising in any axis.

The anisotropic approach can only be applied following biomechanical analysis to determine that greater implant mechanical properties is required in certain axes as opposed to other axes. For example, an implant may be subjected to very high bending forces but only nominal tensile forces and therefore require a much greater emphasis on bending forces. Other relevant axes of force in a medical implant can include tensile, compression, bending, torsion, shear, pull-out (from bone) force, etc.

There are several factors that affect the mechanical properties of an implant. As described above, material composition alone results in a generally uniform or isotropic structure. Without wishing to be limited by a closed list or a single hypothesis, within fiber-reinforced biocomposite medical implants, an anisotropic structure may result from one or more of the following characteristics:
1. The weight ratio of reinforcing fibers to biopolymer. Preferably this ratio is in the range of 1:1 to 3:1 and more preferably 1.5:1 to 2.5:1.
2. The density of the medical implant (this characteristic is also determined to some extent the ratio of reinforcing fiber to polymer)
3. The diameter of reinforcing fiber. The average fiber diameter is preferably between 5 and 50 μm. More preferably between 10-30 μm.
4. Length of fiber (continuous fiber, long fiber, short fiber). Preferably, having continuous fiber reinforcement with fibers that run across the entire implant.
5. The alignment of fibers or fiber layers. Preferably, in each segment of the implant, a majority of fibers or fiber layers are aligned or partially aligned with the axis that will be exposed to the highest bending forces. If partially aligned, then preferably within a 45° angle of the axis.
6. The number of fibers or fiber layers aligned in any given direction. Preferably fiber layers are 0.1 to 1 mm in thickness and more preferably 0.15 to 0.25 mm.
7. The order of fiber layers.

In one embodiment of the present invention, the medical implant is a pin, screw, or wire.

Preferably, a pin or wire of 2 mm external diameter will have a shear load carrying capacity of greater than 200 N. More preferably shear load carrying capacity of 2 mm pin will exceed 400 N and most preferably will exceed 600 N.

Clinical Applications

The medical implants discussed herein are generally used for bone fracture reduction and fixation to restore anatomical relationships. Such fixation optionally and preferably includes one or more, and more preferably all, of stable fixation, preservation of blood supply to the bone and surrounding soft tissue, and early, active mobilization of the part and patient.

There are several exemplary, illustrative, non-limiting types of bone fixation implants for which the materials and concepts described according to at least some embodiments of the present invention may be relevant, as follows:

Screws

Screws are used for internal bone fixation and there are different designs based on the type of fracture and how the screw will be used. Screws come in different sizes for use with bones of different sizes. Screws can be used alone to hold a fracture, as well as with plates, rods, or nails. After the bone heals, screws may be either left in place or removed.

Screws are threaded, though threading can be either complete or partial. Screws can include compression screws, locking screws, and/or cannulated screws. External screw diameter can be as small as 0.5 or 1.0 mm but is generally less than 3.0 mm for smaller bone fixation. Larger bone cortical screws can be up to 5.0 mm and cancellous screws can even reach 7-8 mm. Some screws are self-tapping and others require drilling prior to insertion of the screw. For cannulated screws, a hollow section in the middle is generally larger than 1 mm diameter in order to accommodate guide wires.

Wires/Pins

Wires are often used to pin bones back together. They are often used to hold together pieces of bone that are too small to be fixed with screws. They can be used in conjunction with other forms of internal fixation, but they can be used alone to treat fractures of small bones, such as those found in the hand or foot. Wires or pins may have sharp points on either one side or both sides for insertion or drilling into the bone.

“K-wire” is a particular type of wire generally made from stainless steel, titanium, or nitinol and of dimensions in the range of 0.5-2.0 mm diameter and 2-25 cm length. “Steinman pins” are general in the range of 2.0-5.0 mm diameter and 2-25 cm length. Nonetheless, the terms pin and wire for bone fixation are used herein interchangeably.

Anchors

Anchors and particularly suture anchors are fixation devices for fixing tendons and ligaments to bone. They are comprised of an anchor mechanism, which is inserted into the bone, and one or more eyelets, holes or loops in the anchor through which the suture passes. This links the anchor to the suture. The anchor which is inserted into the bone may be a screw mechanism or an interference mechanism. Anchors are generally in the range of 1.0-6.5 mm diameter

Cable, Ties, Wire Ties

Cables, ties, or wire ties (one example of wire tie is Synthes ZipFix™) can be used to perform fixation by cerclage, or binding, bones together. Such implants may optionally hold together bone that cannot be fixated using penetration screws or wires/pin, either due to bone damage or presence of implant shaft within bone. Generally, diameter of such cable or tie implants is optionally in the range of 1.0 mm-2.0 mm and preferably in the range of 1.25-1.75 mm. Wire tie width may optionally be in the range of 1-10 mm.

Nails or Rods

In some fractures of the long bones, medical best practice to hold the bone pieces together is through insertion of a rod or nail through the hollow center of the bone that normally contains some marrow. Screws at each end of the rod are used to keep the fracture from shortening or rotating, and also hold the rod in place until the fracture has healed. Rods and screws may be left in the bone after healing is complete. Nails or rods for bone fixation are generally 20-50 cm in length and 5-20 mm in diameter (preferably 9-16 mm). A hollow section in the middle of nail or rod is generally larger than 1 mm diameter in order to accommodate guide wires.

Other non-limiting, illustrative examples of bone fixation implants may optionally include plates, plate and screw systems, and external fixators.

Any of the above-described bone fixation implants may optionally be used to fixate various fracture types including but not limited to comminuted fractures, segmental fractures, non-union fractures, fractures with bone loss, proximal and distal fractures, diaphyseal fractures, osteotomy sites, etc.

### Example 1—Modified Surface Area

This non-limiting, illustrative example describes surface treatment with grit blasting of orthopedic implants comprised of reinforced biocomposite materials. This example demonstrates the changes in the surface properties due to described treatment.

Materials & Methods

An ACL interference screw, outer diameter of 9 mm and 30 mm length, was produced using reinforced composite material. Material composite was comprised of PLDLA 70/30 polymer reinforced with 50% w/w continuous mineral fibers. Mineral fibers composition was approximately Na2O 14%, MgO 5.4%, CaO 9%, B₂O₃ 2.3%, P₂O₅ 1.5%, and SiO₂ 67.8% w/w. Testing samples were manufactured by compression molding of multiple layers of composite material into a screw mold. Each layer was comprised of the PLDLA polymer with embedded uni-directionally aligned continuous fibers. Orientation of layers relative to longitudinal axis of implant were 0° (parallel to implant longitudinal axis), 45°, 0°, −45°, 0°, in a repetitive manner according to number of layers in the implant. Each layer was approximately 0.18 mm thick.

Surface was treated by grit blasting using hydroxyapatite grit (approximately 70 μm average diameter) onto the surface of the implants and rotating the implant for complete coverage.

Scanning electron microscope (FEI Helios 600, Holland) was used to image the implant surface. Images were captured at several magnifications, after Au sputtering, and using EDT detectors.

Results

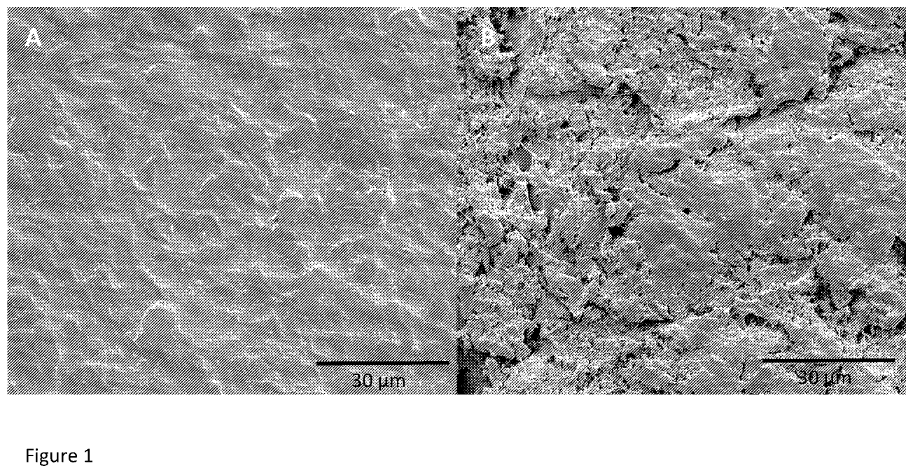

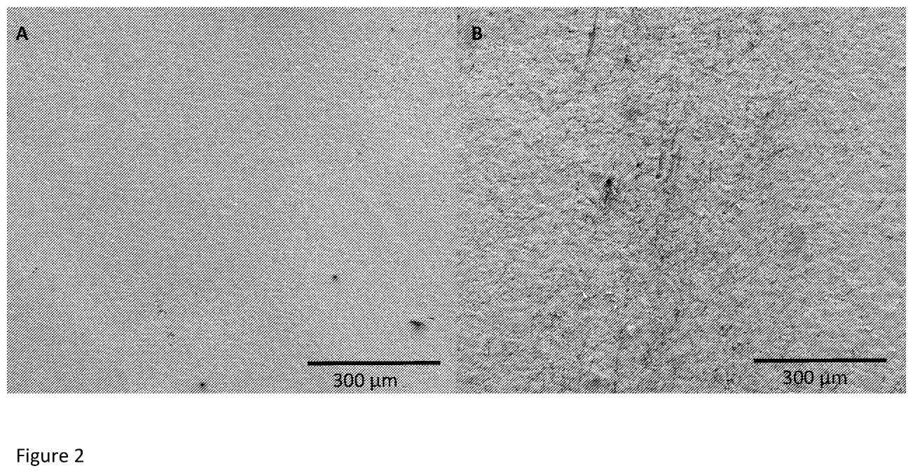

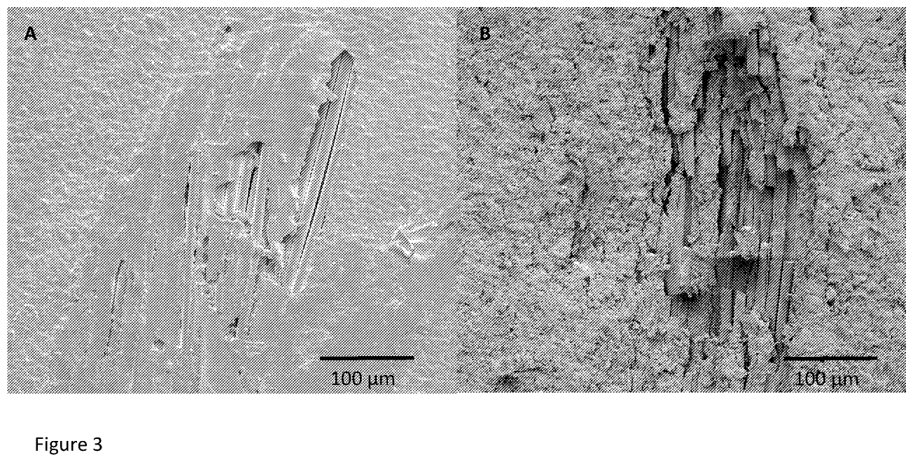

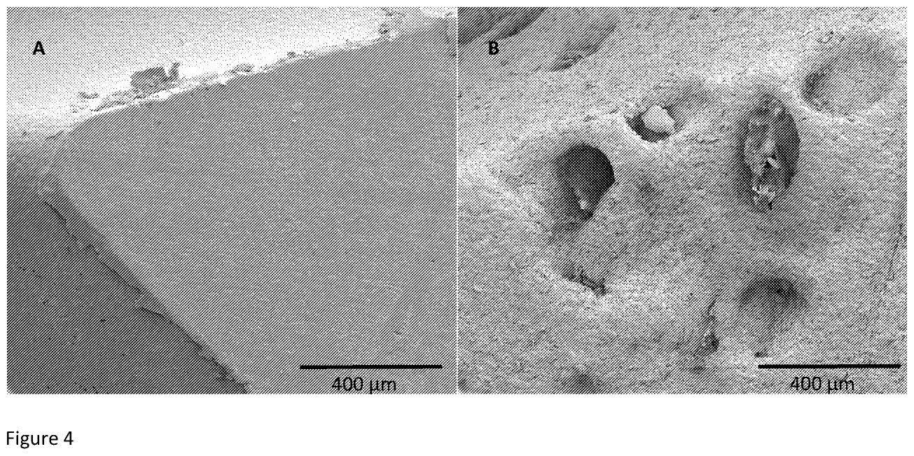

Surface treatment resulted in an increased in the roughness of the surface as can be seen in FIGS. 1 and 2. This roughness can facilitate improved cell attachment and osseo-integration. Due to the compression molding manufacturing technique the outer layer of the implant is mostly smooth polymer and the mineral component that has the bioactive ingredients is not exposed to the cells. This technique facilitates the integration of the cells into the implant due to the morphological change and increases the degradation of the outer layer of the polymer exposing the bioactive minerals at a faster rate, hence again later increasing the osteo-conductive properties of the implant. In addition surface treatment seems to increase the exposure of mineral fibers in the specific locations were fibers are close to the implant surface (FIGS. 3 and 4).

Without wishing to be limited by a single hypothesis, it is believed that surface treatment seen in this example can be a significantly contributing factor to an increase in osseo-integration.

### Example 2—Plates

The below example describes production of thin orthopedic plates with reinforced biocomposite materials. This example demonstrates the different surface properties of medical implant plates comprised of reinforced biocomposite materials due to surface treatments.

Materials & Methods

Plate implants, each with a thickness of 2 mm, width of 12.8 mm and 6 cm length were produced using reinforced composite material. Material composite was comprised of PLDLA 70/30 polymer reinforced with 50% w/w continuous mineral fibers. Mineral fibers composition was approximately Na₂O 14%, MgO 5.4%, CaO 9%, B₂O₃ 2.3%, P₂O₅ 1.5%, and SiO₂ 67.8% w/w. Testing samples were manufactured by compression molding of multiple layers of composite material into a rectangle mold. Each layer was comprised of the PLDLA polymer with embedded uni-directionally aligned continuous fibers. Orientation of layers relative to longitudinal axis of implant were 0° (parallel to implant longitudinal axis), 45°, 0°, −45°, 0°, in a repetitive manner according to number of layers in the implant. Each layer was approximately 0.15 mm thick. Plates were either not treated or treated by blasting using hydroxyapatite grit onto the surface of the implants while rotating the implant for complete coverage under three different blasting conditions.

Scanning electron microscope (FEI Quanta FEG 250, Holland) was used to image the implant surface. Images were captured at several magnifications using EDT& BS detectors. Samples were cut, cross-sections were imaged by the SEM and surface layer thickness was measured based on distance of mineral fibers from implant edge by the SEM program.

SEM-EDS was used for elemental analysis and data was compared between conditions using 15 Kv and a magnification of ×500.

A Focused ion beam setup (FIB) (Helios 600, FEI) was also used to carve a hole and image the cross-section in a treated implant which was coated with Au prior to carving.

Atomic force microscopy (AFM) was used to characterize the surface roughness and surface area increase. AFM measurements were done by using ICON (Bruker, USA) and Bio FastScan (Bruker) Tapping mode, silicon probe TESP (Bruker), spring constant 20-80 N/m, freq. 279-389 kHz.

Results

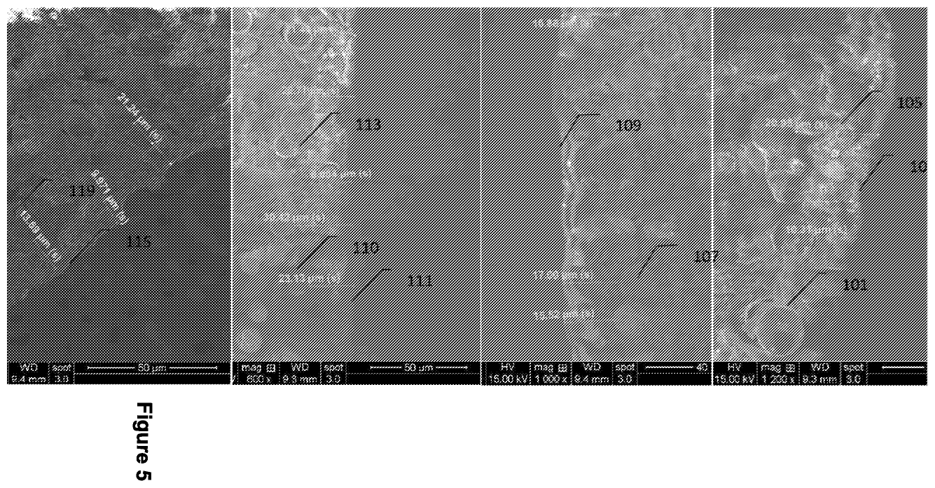

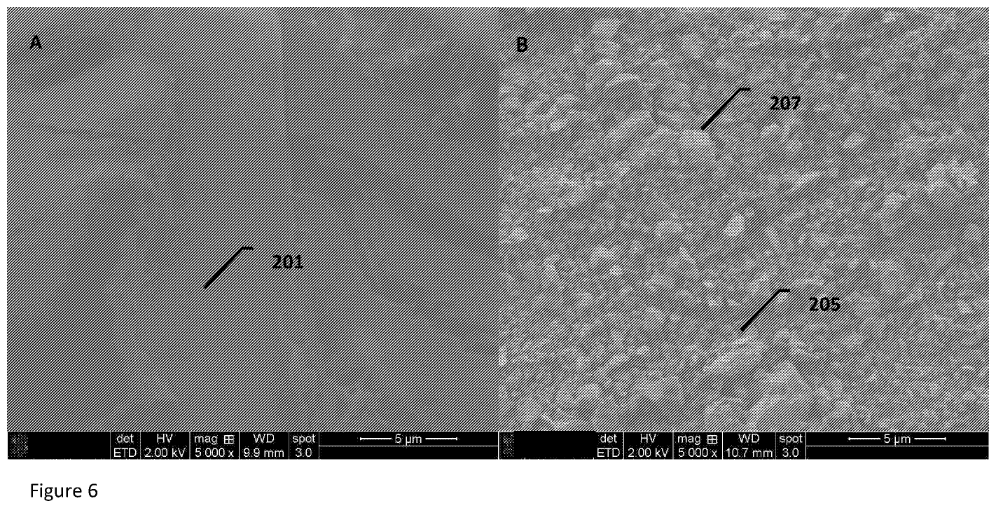

Due to the compression molding manufacturing technique the outer layer of the implant is mostly smooth polymer and the mineral component that has the bioactive ingredients which makes up the body of the implant, is not exposed to the cells. In such an implant example the surface layer, defined as the outer mostly polymer layer, was measured to be 17.6±6.8 micron (FIG. 5). Surface treatment resulted in an increased in the roughness of the surface as can be seen in FIGS. 6A and 6B. In 201 the surface is relatively smooth in compare to 205 and 207. This roughness can facilitate improved cell attachment and osseo-integration. This blasting technique facilitates the integration of the cells into the implant due to the morphological change and increases the degradation of the outer layer of the polymer exposing the bioactive minerals at a faster rate, hence again later increasing the osteo-conductive properties of the implant. In addition, surface treatment increases surface area by up to 64% (Table 1). Roughness and roughness max was increased from 35.8 nm to 433 nm and from 0.357 micron to 5.2 micron due to treatments, respectively. Table 1 summarizes the increased roughness parameters both Ra, Ra max and surface areas for three treatments.

TABLE 1

| | Roughness (Ra) | Roughness Rmax | Surface area |
|---|---|---|---|
| Condition | [nm] | [um] | Difference |
| No treatment (A) | 35.8 | 0.357 | 0.13% |
| Treatment 1 (B) | 433 | 2.287 | 17% |
| Treatment 2 (C) | 326 | 3.5 | 64.2% |
| Treatment 3 (D) | 388 | 5.2 | 58.3% |

Specifically, Table 1 shows surface roughness measurements done with an Atomic force microscope (AFM) (ICON (Bruker, USA) and Bio FastScan (Bruker, USA) representing (A) the surface of the implant as it comes out of the mold (B, C, D) the surface after blasting with three different conditions. AFM measurements were done by using ICON (Bruker, USA) and Bio FastScan (Bruker), tapping mode, silicon probe TESP (Bruker), spring constant 20-80 N/m, freq. 279-389 kHz.

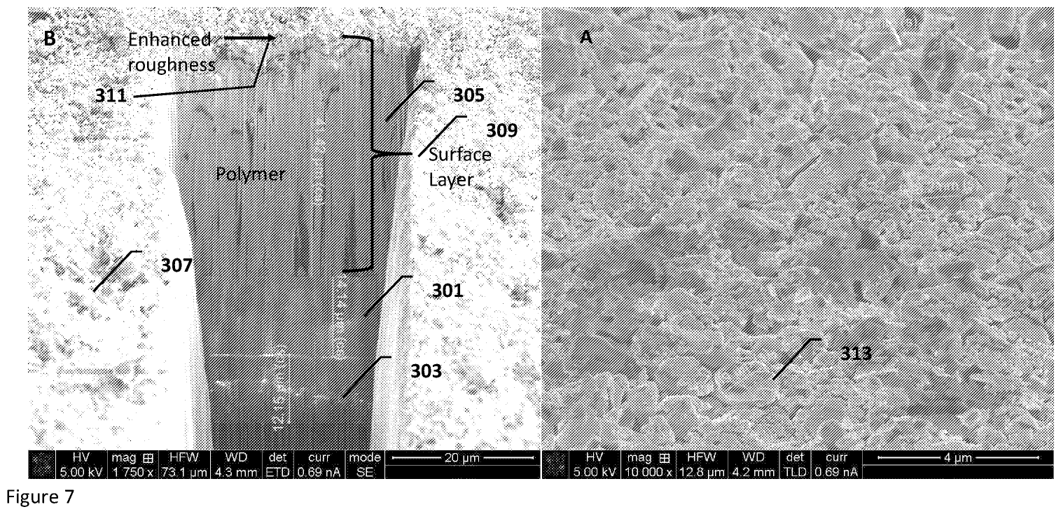

A hole was carved into an implant using a FIB setup exposing an inner crossection of more than 60 micron deep (FIGS. 7A and 7B). In this case an outer surface layer was observed as approximately 2.5 micron (311) and the inner surface layer as an additional 40 micron (305). The cross-section of two overlapping mineral fibers can be seen in this image as well (301, 303). They represent the edge of the body section of the implant. The roughness on the surface in 307 was zoomed in to reveal features around 1 micron in diameter (313).

Elemental composition differences were noted between the implant body, implant inner surface layer and outer surface layer (Table 2). Specifically a decrease in mineral content can be seen between the inner surface and the body of the implant. A decrease in Si content can be seen in the outer surface vs the body of the implant. In this case the phosphate and calcium concentrations are significantly higher in the outer surface layer and not detected in both the inner surface layer as well as the implant body. In this case the body implant composition was also characterized as having more sodium than the surface area, both inner and outer.

TABLE 2

| | A Implant body | | B Inner Surface Layer | | C Outer Layer | |
|---|---|---|---|---|---|---|
| | (Mld Implant) | | (No Treatment) | | (SF1) | |
| Element | Wt % Avg | At % Avg | Wt % Avg | At % Avg | Wt % Avg | At % Avg |
| C | 43.71 ± 4.62 | 54.33 ± 4.34 | 51.48 ± 2.5 | 59.57 ± 2.1 | 19.74 ± 1.55 | 31.27 ± 1.375 |
| O | 37.998 ± 1.4 | 35.57 ± 2.24 | 43.48 ± 0.77 | 37.79 ± 1.14 | 39.91 ± 2.41 | 47.49 ± 1.21 |
| Na | 4.54 ± 0.77 | 2.97 ± 0.58 | 1.39 ± 0.42 | 0.84 ± 0.27 | 0.257 ± 0.44 | 0.21 ± 0.36 |
| Mg | 0.98 ± 0.16 | 0.6 ± 0.12 | 0.157 ± 0.27 | 0.09 ± 0.16 | 0.42 ± 0.1 | 0.32 ± 0.06 |
| SI | 10.84 ± 2.26 | 5.8 ± 1.37 | 3.32 ± 1.07 | 1.65 ± 0.55 | \ | \ |
| P | \ | \ | \ | \ | 12.77 ± 0.81 | 7.87 ± 0.76 |
| Ca | 1.92 ± 0.24 | 0.72 ± 0.1 | 0.17 ± 0.3 | 0.06 ± 0.1 | 26.91 ± 3.62 | 12.84 ± 2.16 |

| | D Outer Layer | | E Outer Layer | |
|---|---|---|---|---|
| | (SF2) | | (SF3) | |
| Element | Wt % Avg | At % Avg | Wt % Avg | At % Avg |
| C | 30.19 ± 2.15 | 42.66 ± 2.12 | 25.78 ± 3.9 | 38.1 ± 4.17 |
| O | 41.4 ± 1.75 | 43.95 ± 1.59 | 39.88 ± 1.56 | 44.38 ± 0.42 |
| Na | 0.14 ± 0.25 | 0.11 ± 0.2 | 0.7 ± 0.9 | 0.56 ± 0.725 |
| Mg | 0.32 ± 0.05 | 0.22 ± 0.03 | 0.46 ± 0.14 | 0.34 ± 0.11 |
| SI | 0.28 ± 0.48 | 0.17 ± 0.3 | 2.55 ± 3.715 | 1.67 ± 2.45 |
| P | 9.1 ± 0.75 | 5 ± 0.52 | 9.58 ± 0.76 | 5.5 ± 0.52 |
| Ca | 18.57 ± 1.82 | 7.89 ± 0.95 | 20.79 ± 1.5 | 9.26 ± 0.985 |

Table 2 shows that energy-dispersive X-ray spectroscopy (EDS) measurements of elemental composition representing (A) the center of the implant cross-section, (B) the surface of the implant as it comes out of the mold (C, D, E) the surface after three different treatments which increases roughness and creates small nm and micron holes, which facilitate cell in-growth and degradation.

### Example 3—Small Diameter Pins

This Example describes production of small diameter orthopedic pins with reinforced biocomposite materials. This example demonstrates how medical implant pins comprised of reinforced biocomposite materials can have surface areas of several compositions.

Materials & Methods

Pin implants, each of outer diameter 2 mm and 7 cm length, were produced using reinforced composite material. Material composite was comprised of PLDLA 70/30 polymer reinforced with 50% w/w. Mineral fibers composition was approximately Na₂O 14%, MgO 5.4%, CaO 9%, B₂O₃ 2.3%, P₂O₅ 1.5%, and SiO₂ 67.8% w/w. Testing samples were manufactured by compression molding of multiple layers of composite material into a multi-tubular mold. Each mold was designed to create simultaneously 14 implants. Each layer was comprised of the PLDLA polymer with embedded uni-directionally aligned continuous fibers. Orientation of layers relative to longitudinal axis of implant were 0° (parallel to implant longitudinal axis), 45°, 0°, −45°, 0°, in a repetitive manner according to number of layers in the implant. Each layer was approximately 0.15 mm thick. After extraction of the pins from the molds Computer Numerical Control (CNC) machining was used to separate the 14 pins and to create an angled tip.

Scanning electron microscope (SEM) (FEI Quanta FEG 250, Holland) images were captured for surface and for cross-sections of implant samples at several magnifications and using either EDT or BSE detectors. ImageJ™ (NIH Image Processing Software) was used to measure the surface percentage differences based on the circumference.

SEM-EDS was used for elemental analysis and data was compared between conditions using 15 Kv and a magnification of ×500.

Results

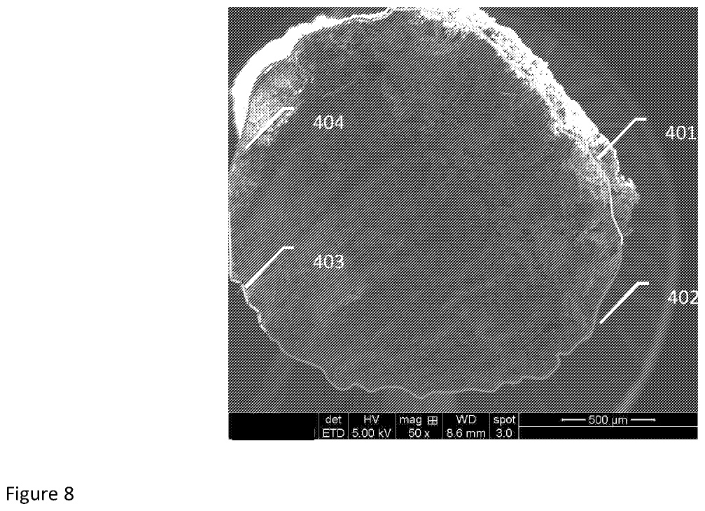

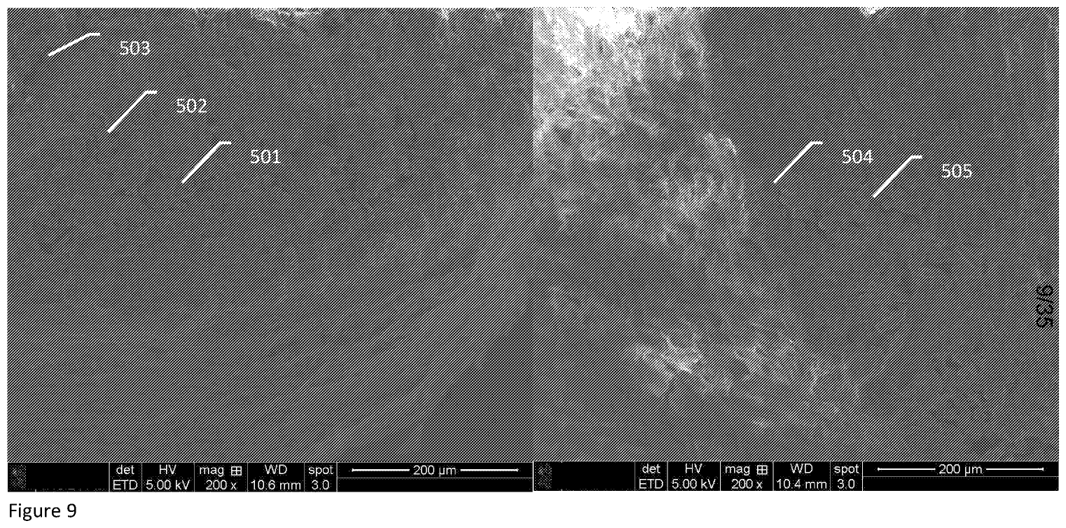

Computer Numerical Control (CNC) machining which separated the pins was used to expose the inner content of the biocomposite, creating a pin implant which has a surface layer of less than 60% with a different composition than the inner body (FIG. 8). Table 3 provides a key to the drawing.

TABLE 3

| | | % of |
|---|---|---|
| Label | L (μm) | Circumference |
| Surface layer with different | 4073.5 | 59% |
| composition (Red, 404 + 402) | | |
| Exposed inner content on surface | 2863.9 | 41% |
| (Green, 403 + 401) | | |

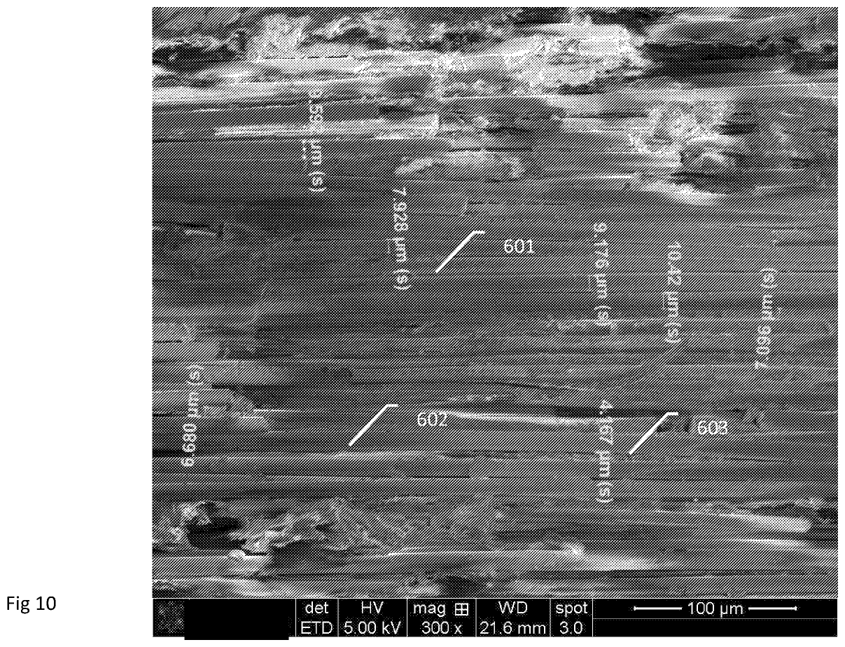

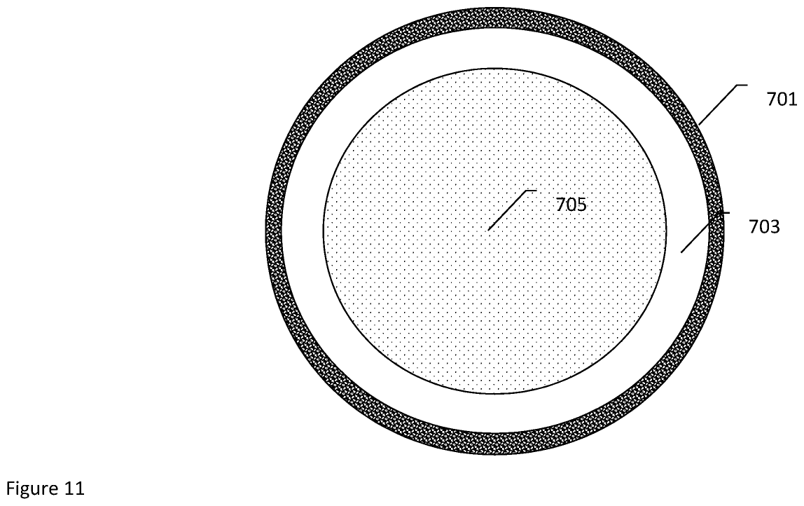

CNC machining treatment to the tip (FIG. 9) resulted in partially exposed fiber bundles (501-507). Exposed fibers can be seen due to the treatment, these facilitate cell in-growth. Continuous fiber body composition can be seen in FIG. 10, which include representative measurements of fiber diameters, as seen in 601-602 and distances between fibers (603). This body composition is an example of ˜1:1 mineral to polymer w/w ratio. FIG. 11 shows schematics of a representative pin implant cross-section when created in a single-tubular mold (unlike the above multi-tubular mold) followed by the blasting treatment described in previous example. Schematics are not in scale, but include the implant body comprised of one composition, 705, the surface layer which in this case includes both an inner surface layer 703 and an outer surface layer 701, each with a different composition. This method results in an outer surface layer which is entirely of a different composition than the inner body.

Laser Cut Ablation

Examples 4 and 5 relate to a surface treated implant, in which the surface was treated with precise laser cut ablation. When examining the cross-section of a biocomposite medical implant, a layer of uniform biopolymer (PLDLA), without the presence of the reinforcing fibers, can be measured with varying thickness.

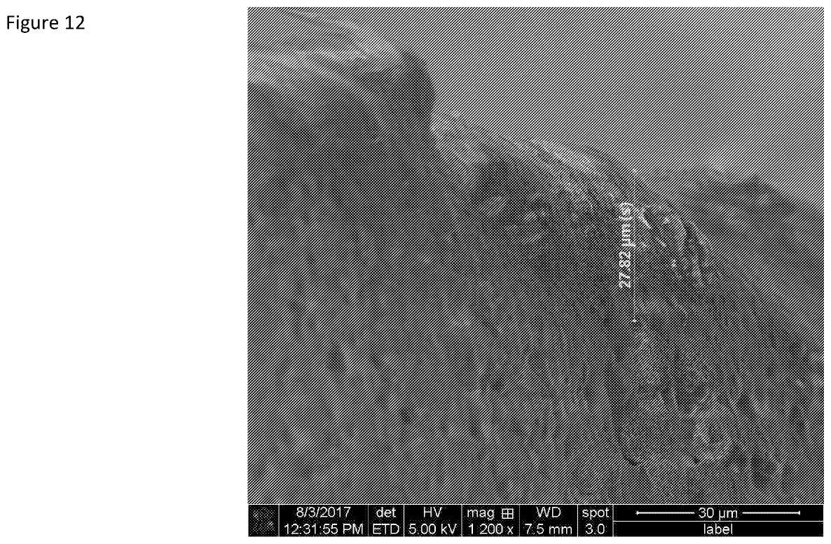

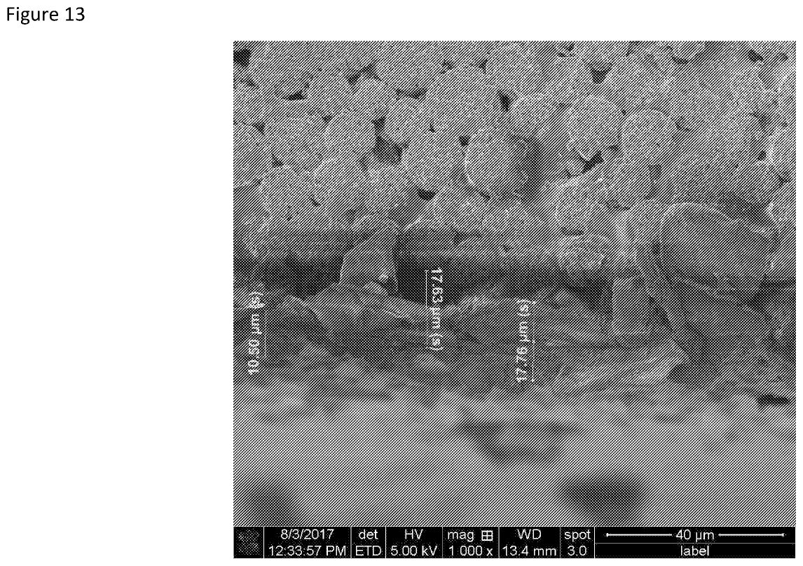

FIGS. 12 and 13 depict different biopolymer layer thickness at different medical implants cross sections.

The biocomposite medical implant performance can be affected by the outer layer surface characteristics. For example, but not limited to, these characteristics of the outer layer surface:

1. Percentage of exposed fibers.

2. Outer layer thickness.

3. Exposed fibers directional/multi-directional orientation.

4. Fiber exposure type—cross-sectional and/or circumferential

5. Treated to untreated surface ratio.

6. Surface treatment pattern shape.

7. Surface treatment pattern repetition.

8. Surface roughness.

9. Resultant surface geometry after surface treatment.

10. Material composition of the outer layer.

Medical Implant Outer Layer Surface Thickness

When examining the cross-section of the medical implant, a layer of uniform biopolymer (PLDLA), without the presence of the reinforcing fibers, can be measured with varying thickness.

Optionally the outer surface layer of uniform biopolymer varies across different cross-sections of the medical implant.

Also optionally, the outer surface layer of uniform biopolymer is thicker on the implant body comparing to the layer thickness on the ribs/threads.

Also optionally, the outer surface layer of uniform biopolymer is thicker on one side of the medical implant comparing to the opposite side.

Also optionally, the outer surface layer of uniform biopolymer is thicker on the implant body comparing to the layer thickness on the implant circumference.

The outer surface layer thickness may also change per design and/or surface angles and/or surface nooks and crannies presence.

Optionally, the outer surface layer of uniform biopolymer is more than 3 microns.

Also optionally, the outer surface layer of uniform biopolymer is more than 10 microns (see Example 4).

Also optionally, the outer surface layer of uniform biopolymer is more than 25 microns.

FIGS. 12 and 13 depict different biopolymer layer thickness at different medical implants cross sections.

Treated Surface Physical Characteristics

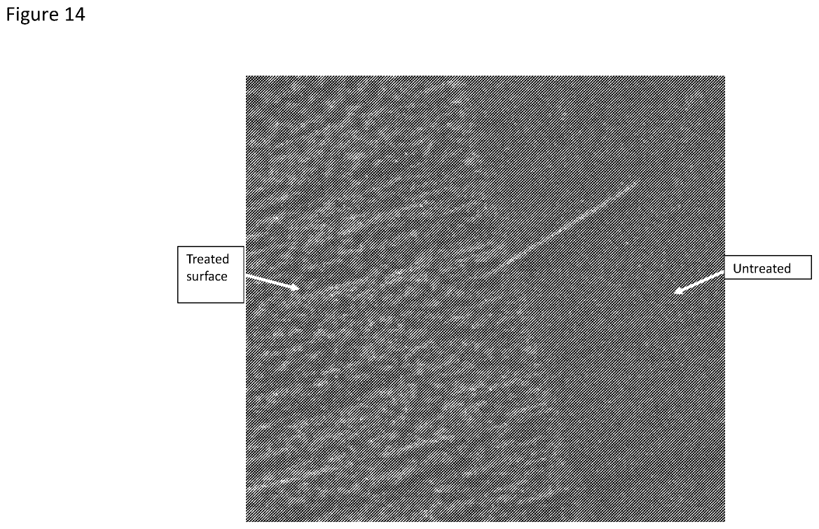

FIG. 14 shows top view of untreated vs. treated surface layer.

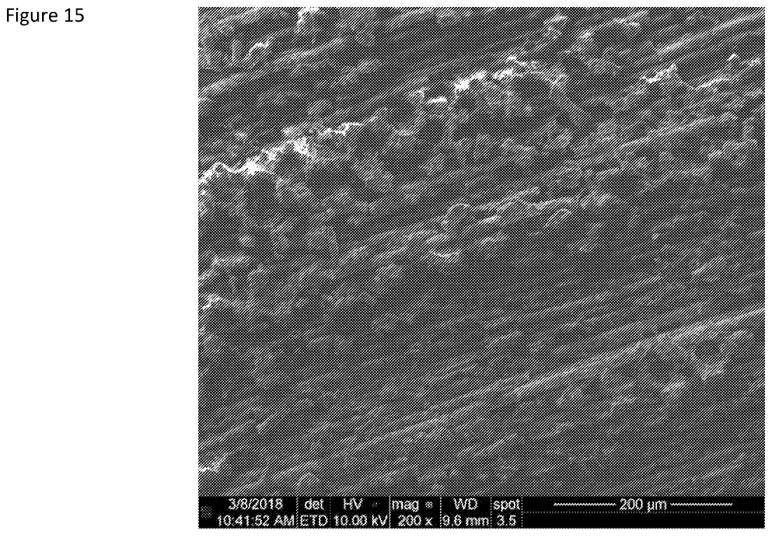

The untreated surface layer is a uniform layer of biopolymer and the treated surface has visual fibers exposed. FIG. 15 shows a magnified view of the surface treated section, with clear view of the exposed fibers and of some biopolymer residues.

Treated/Untreated Surface Location

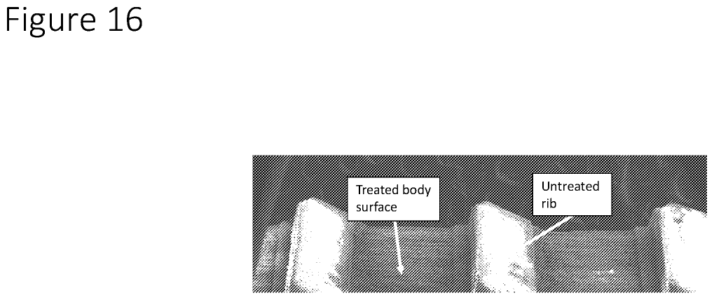

Depending on the medical implant design, different regions of the implant may undergo surface treatment. Optionally, the medical implant body will be surface treated completely while the implant ribs and/or threads remains untreated (see Example 4). Also optionally, the medical implant body will be partially surface treated while the implant ribs and/or threads remains untreated. Also optionally, the medical implant body will be partially surface treated while the implant circumference remains untreated. Also optionally, the medical implant body will remain untreated while the implant circumference will be surface treated (see Example 5). Also optionally, the medical implant body surface will remain untreated while the implant ribs and/or threads will be surface treated. Also optionally, the medical implant body will be partially surface treated while the implant ribs and/or threads remains untreated. FIG. 16 shows one example of treated and untreated area of the medical implant.

Treated/Untreated Surface Ratio

Depending on the medical implant design, sections of different sizes, might undergo surface treatment. That is, the ratio between the treated surface area to the entire medical implant surface area may vary.

Optionally, 10-70% of the medical implant outer surface will be surface treated. (see Example 5). Also optionally, 30-55% of the medical implant outer surface will be surface treated. Also optionally, 20-45% of the medical implant outer surface will be surface treated (see Example 4).

Treated Surface Pattern Shape

Depending on the medical implant design, different section shapes will be surface treated. The medical implant surface treatment shape may be: rectangular (see Example 4), circular, arc shaped, diamond, parallelograms, triangular or any other combination of these shape.

The surface treatment shape may also be a line shape of specified width. The line type may be: continuous solid line, dashed line, dotted line, circumferential line (see Example 5), angled line (any angle from 5 to 85 degrees), helix line (helix angle of 5 to 85 degrees). Surface treated line width can optionally be more than 5 microns. Also optionally, surface treated line width can optionally be more than 10 microns. Also optionally, surface treated line width can optionally be more than 20 microns.

Treated Surface Pattern Repetition

The surface treated pattern may appear in different repetition types (see Example 4). Optionally, the treated surface shape will be repeated over the entire treated area on the medical implant. Also optionally, the entire surface treated area of the medical implant will be line treatment pattern. Also optionally, more than one surface treated shapes will appear on the treated surface area of the medical implant. Also optionally, more than one line treatment pattern will appear on the treated surface area of the medical implant. Also optionally, a combination of one or more line treatment pattern and one or more shape surface treatment pattern may appear.

Directional Fiber Exposure

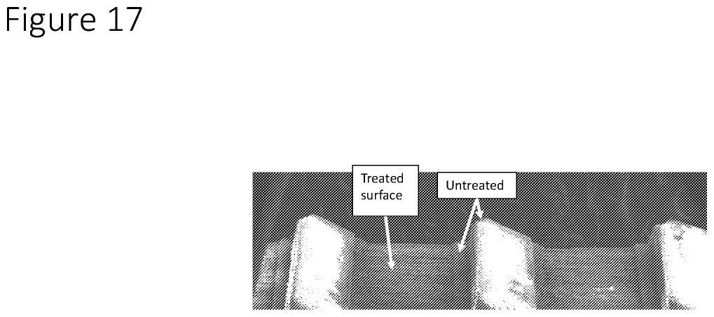

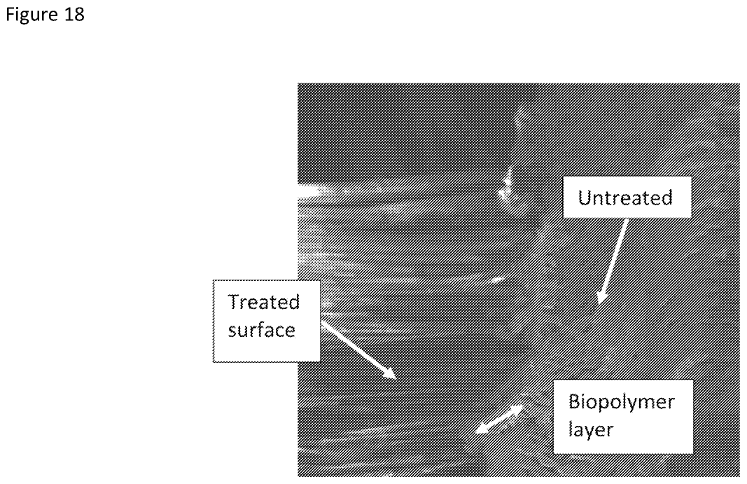

The surface treatment can expose fibers of different orientations on the outer layer surface. Optionally, the exposed fiber orientation is parallel to the medical implant body axis (see Example 4). Also optionally, the exposed fiber orientation is 5°-85° relative to the implant body axis. Also optionally, the exposed fiber orientation is 15°-65° relative to the implant body axis. Also optionally, the exposed fiber orientation is 30°-60° relative to the implant body axis. FIGS. 17 and 18 show directional fiber orientation exposure of two different samples.

Multi-Directional Fiber Exposure

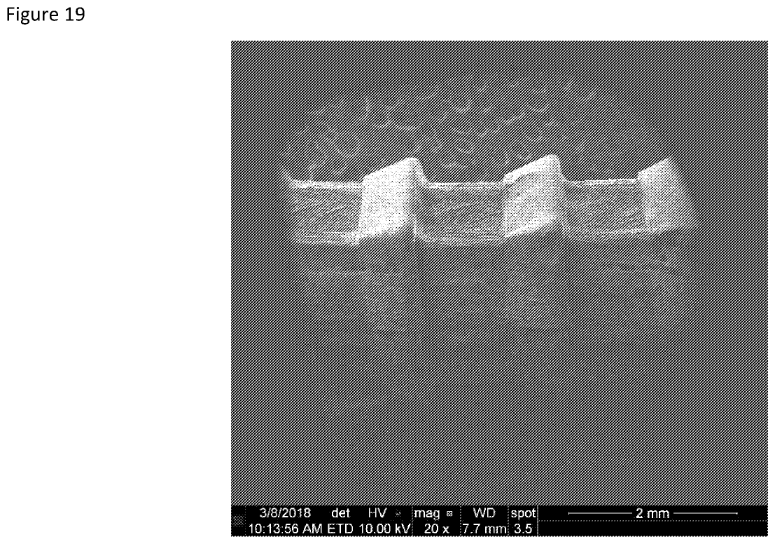

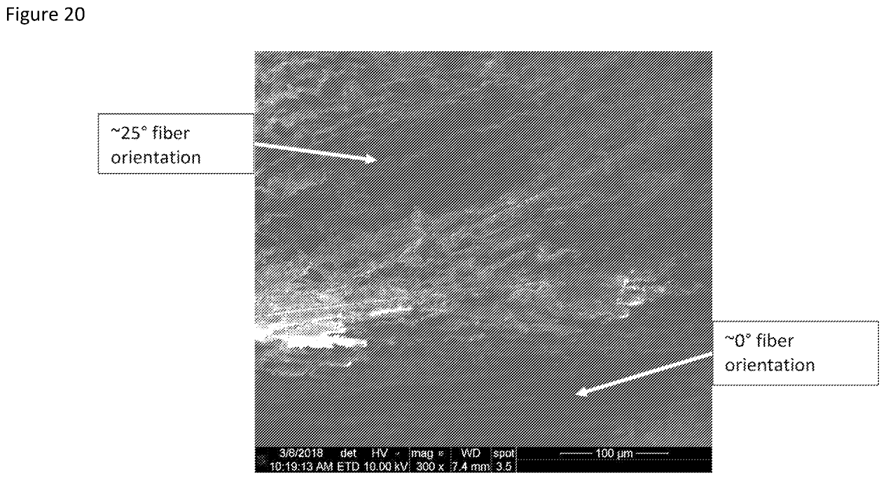

Fibers exposure can also be achieved for unparallel fiber orientation on the outer surface of the medical implant. Meaning, a combination of more than one fiber directions exposure on the same surface. Optionally, there is more than 1 exposed fiber direction on the treated surface. Also optionally, there are more than 2 exposed fiber directions on the treated surface. Also optionally, the angle between one area of fiber direction to its neighboring area with different fiber direction is between 0°-90°. Also optionally, the angle between one area of fiber direction to its neighboring area with different fiber direction is between 25°-75°. FIGS. 19 and 20 depict surface treatment for fiber exposure of a surface which has two different fiber directions.

Fiber Cross-Section Exposure

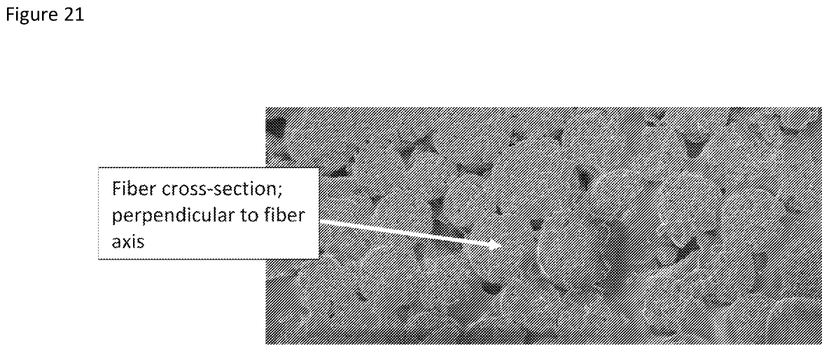

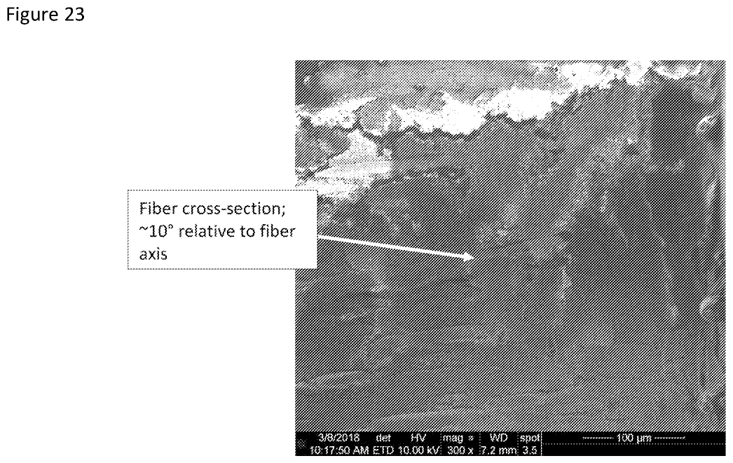

Fiber exposure can be achieved by surface treatment which will intentionally create cutting of the fibers in different angle. This will expose the fibers cross-section as the outer layer surface of the medical implant and can potentially improve implant performance. Optionally, cross-sectional fiber exposure is 90° relative to the fiber axis. Also optionally, cross-sectional fiber exposure is 15°-65° relative to the fiber axis (see Example 5). Also optionally, the cross-sectional fiber exposure comprises of more than one fiber directions. FIGS. 21, 22 and 23 show cross-sectional fibers exposure ˜90°, ˜45° and ˜10° relative to the fiber axis respectively.

Outer Layer Surface Geometry/Roughness

Prior to surface treatment the biocomposite medical implant outer surface geometry is obtained smooth and with no exposed fibers. Surface treatment can achieve several different surface geometries, depending on the ablation method, number of treatment iterations per surface and depending on surface angle relative to the surface treatment application.

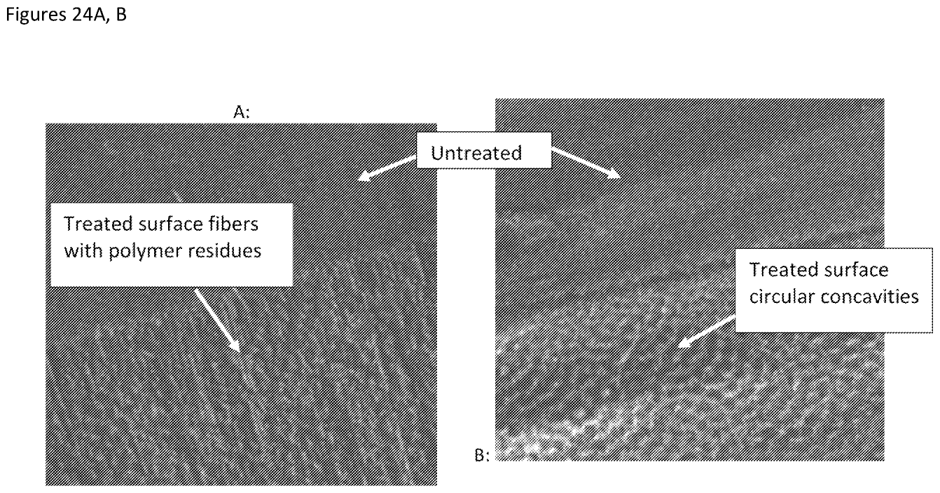

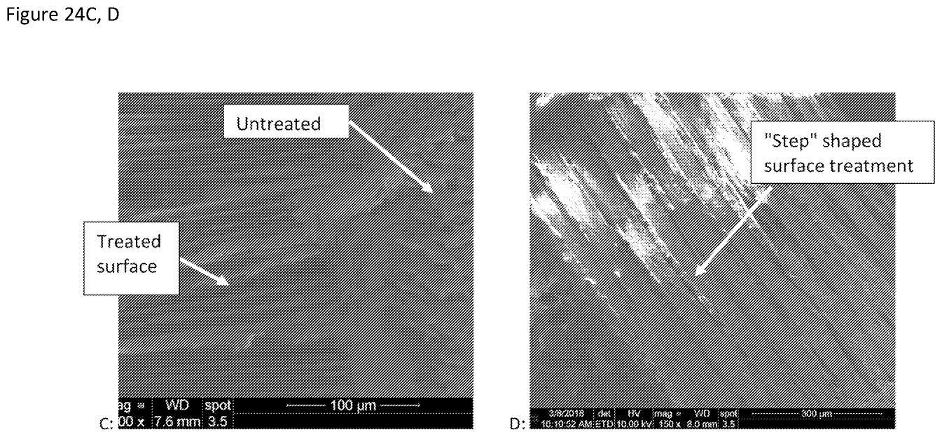

FIGS. 24A-D show four different surface roughness and geometries obtained by surface treatment on biocomposite medical implant. Optionally a surface of said implant is treated to partially expose inner composition. Also optionally, the surface maximum roughness is more than 2 microns. Also optionally, the surface maximum roughness is more than 3 microns. Also optionally, the surface maximum roughness is more than 5 microns.

Without wishing to be limited by a closed list, the surface treatment may result in different kinds of surface geometries. Optionally, the resultant surface geometry is circular concavities. Also optionally, the resultant surface geometry is comprised of the exposed fibers alone. Also optionally, the resultant surface geometry is comprised of the exposed fiber and biopolymer residues (see Example 5). Also optionally, the resultant surface geometry is step shaped (see Example 4).

Outer Layer Material Composition

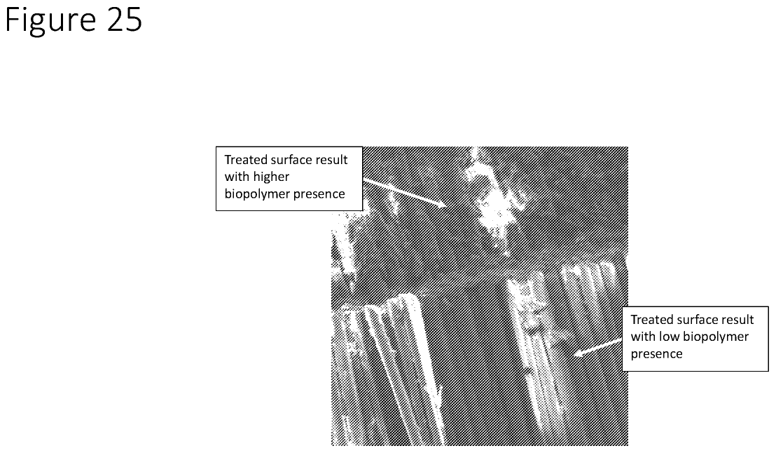

Outer layer surface material composition can be controlled using the surface treatment. FIG. 25 shows top view of two neighboring regions with different outer surface composition as a result of different surface treatments. The top region has higher biopolymer percentage and less exposed fibers while the bottom section has highly exposed fibers with almost no biopolymer presence. Both sections have directional fiber exposure.

### Example 4—Regional Surface Treatment of Hexagonal Ribbed Pin Implant

The example below describes a surface treatment process using laser ablation method on a hexagonal ribbed orthopedic pin implant produced from reinforced biocomposite material. This example details the area and shape of ablation, effect of the ablation on biopolymer layer removal and on directional fibers exposure.

Materials & Preparations

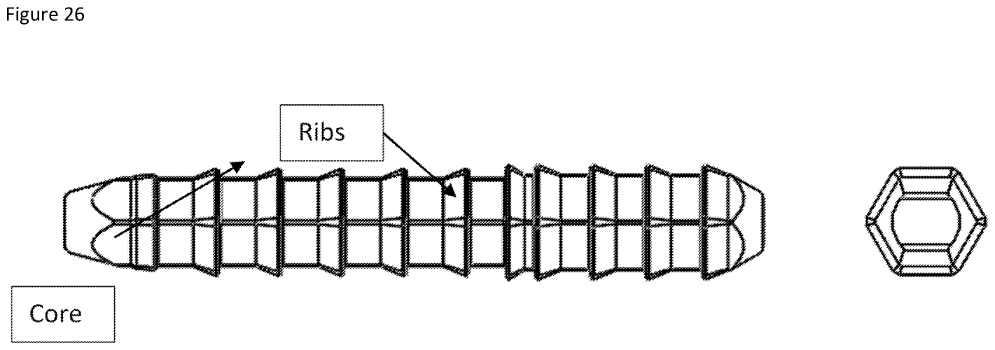

Hexagonal ribbed pin implant was produced using reinforced composite material. The pin has two sides, each with a different hexagonal core cross-section size, 2.4 mm and 2.6 mm. The total length of the pin is 19 mm. The implant ribs are also hexagonal shaped and protrude approximately 0.3 mm from the pin core surface (FIG. 26).

Material composite was comprised of PLDLA 70/30 polymer reinforced with 50% w/w, 70%, or 85% w/w continuous mineral fibers. Mineral fibers composition was approximately Na₂O 14%, MgO 5.4%, CaO 9%, B₂O₃ 2.3%, P₂O₅ 1.5%, and SiO₂ 67.8% w/w. Testing samples were manufactured by compression molding of multiple layers of composite material into a designated single cavity mold. Each layer was comprised of the PLDLA polymer with embedded uni-directionally aligned continuous fibers. Orientation of layers relative to longitudinal axis of implant were 0°. Each layer was approximately 0.18 mm thick.

The implant was molded under controlled environment and kept in a clean state throughout the process stages. The laser ablation of the implant surface was conducted using a high frequency laser machine. The laser machine environment was confined within a laminar airflow hood, all surfaces and jigs were cleaned using alcohol. Particles created by the laser process were vacuumed out of the controlled region.

Methods

Implant Position

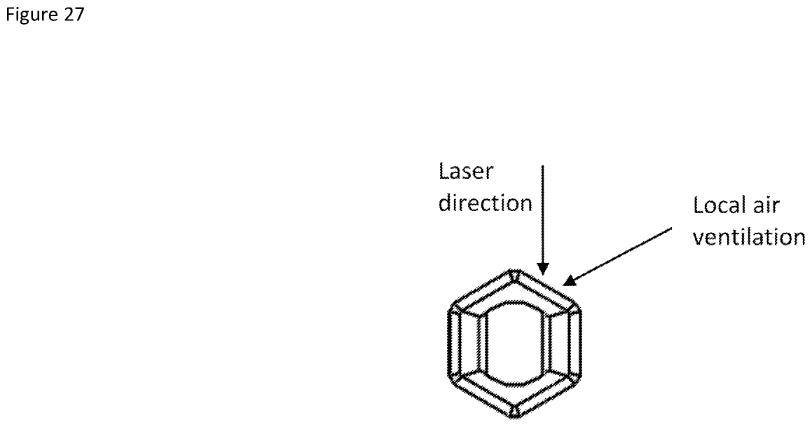

The implant was placed inside a designated jig, at a position as shown in FIG. 27 below. The jig keeps the implant at the desired position relative to the laser application direction. During the laser ablation process, the implant was static and local air ventilation was constantly applied on the ablation position, to prevent local overheating or burn marks from happening.

Laser Ablation Position, Shape and Repetition

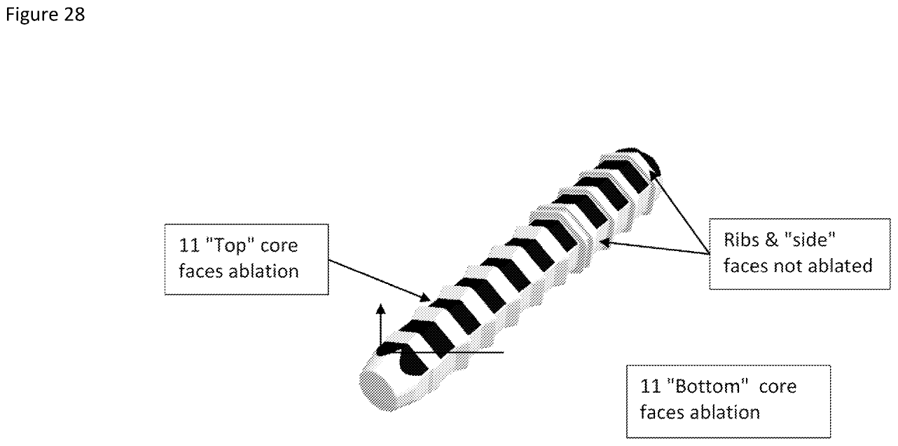

The laser ablation was conducted on four (4) out of six (6) faces of each hexagonal core section of the implant, i.e. two (2) core faces repeated 11 times per position of the implant and 22 faces total (top and bottom side of the implant). FIG. 28 depicts an illustration of this surface ablation selection. An example of such regional surface treatment is shown in FIG. 26.

The total ablated surface area for this implant was approximately 44.1 mm\hat{ }2. For the defined laser ablation surfaces, the ratio between the ablated surface area to the whole implant surface area is approximately 0.22.

Laser Ablation Application—Direction, Laser Focal Point, Iterations

The laser ablation was conducted parallel to the implant longitudinal axis. The implant was molded with orientation of layers relative to longitudinal axis of 0°. This method of laser ablation on the implant core surfaces results in directional fibers exposure. An example of such directional fiber exposure is shown in FIGS. 17 and 18.

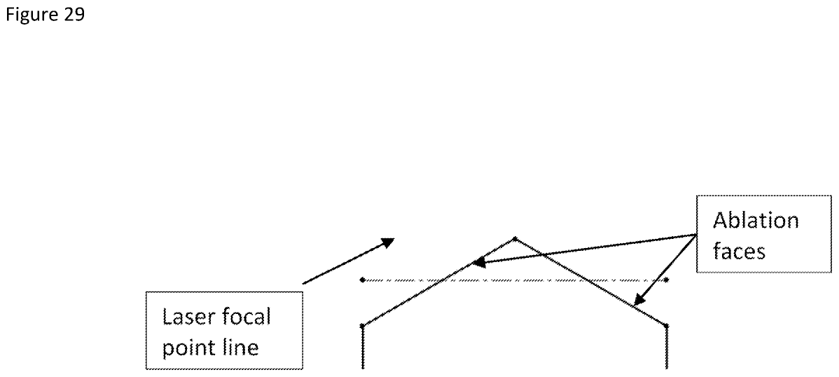

Laser application was conducted with one laser pass per ablated surface. The laser focal point was determined at an intermediate point on the diagonal line of the hex face, see illustration in FIG. 29. The laser ablation on the ablated hex face was divided equally to several parallel application surfaces, division which effectively created a “steps” surface geometry on the ablated surface. FIG. 24D shows an example of such resultant surface geometry.

Results

The result on the implant core is a surface with highly exposed fibers, with the outer biopolymer layer removed. The implant ribs keep their original geometry and original outer layer. Specifically for this implant surface ablation, the core size is reduced by 0.04 mm, i.e. 0.02 mm of uniform biopolymer layer from each side of ablation. FIG. 12 shows an example of uniform biopolymer outer layer with thickness similar to that of the measured sample.

### Example 5—Circumferential Surface Treatment of Hexagonal Ribbed Pin Implant

The example below describes a surface treatment process using laser ablation method on a hexagonal ribbed orthopedic pin implant produced from reinforced biocomposite material. This example details the area and shape of ablation, effect of the ablation on biopolymer layer removal and on fibers cross-section exposure.

Materials & Preparations

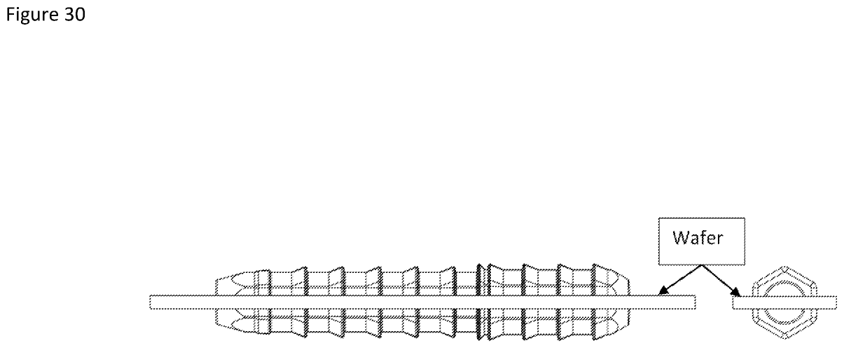

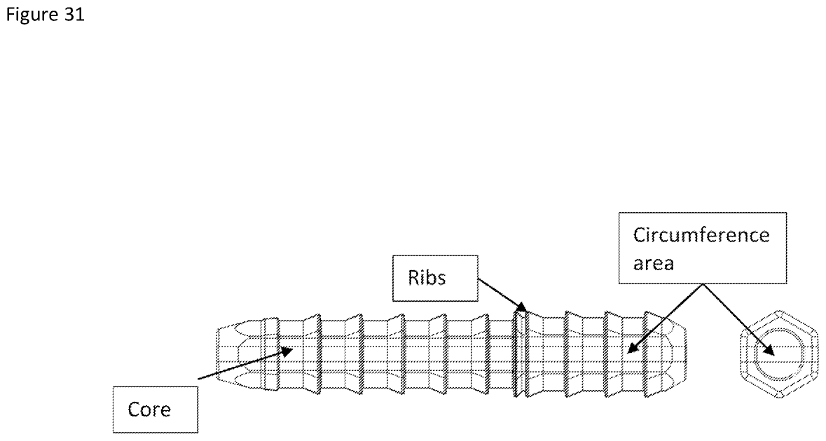

Hexagonal ribbed pin implant was produced using reinforced composite material. The pin has two sides, each with a different hexagonal core cross-section size, 2.4 mm and 2.6 mm. The total length of the pin is 19 mm. The implant ribs are also hexagonal shaped and protrude approximately 0.3 mm from the pin core surface (FIG. 30). This implant configuration was molded with a circumferential wafer attached to the implant, a wafer which later will be removed, exposing the fibers at the desired place and direction relative to the implant axis. The implant after wafer removal is shown in FIG. 31.

Material composite was comprised of PLDLA 70/30 polymer reinforced with 50% w/w, 70%, or 85% w/w continuous mineral fibers. Mineral fibers composition was approximately Na₂O 14%, MgO 5.4%, CaO 9%, B₂O₃ 2.3%, P₂O₅ 1.5%, and SiO₂ 67.8% w/w. Testing samples were manufactured by compression molding of multiple layers of composite material into a designated single cavity mold. Each layer was comprised of the PLDLA polymer with embedded uni-directionally aligned continuous fibers. Orientation of layers relative to longitudinal axis of implant were 0° and 25°. Each layer was approximately 0.18 mm thick.

The implant was molded under controlled environment and kept in a clean state throughout the process stages. The laser ablation of the implant circumference was conducted using a high frequency laser machine. The laser machine environment was confined within a laminar airflow hood, all surfaces and jigs were cleaned using alcohol. Particles created by the laser process were vacuumed out of the controlled region.

Methods

Implant Position

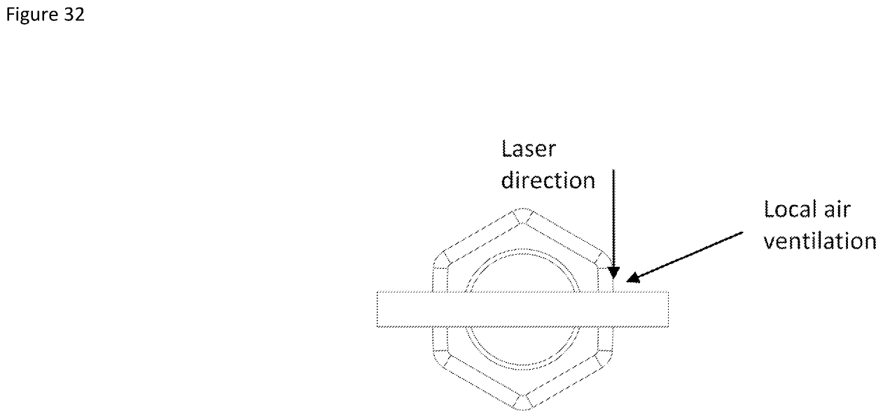

The implant was placed inside a designated jig, at a position as shown in FIG. 32 below. The jig keeps the implant at the desired position relative to the laser application direction. During the laser ablation process, the implant was static and local air ventilation was constantly applied on the ablation position, to prevent local overheating or burn marks from happening.

Laser Ablation Position

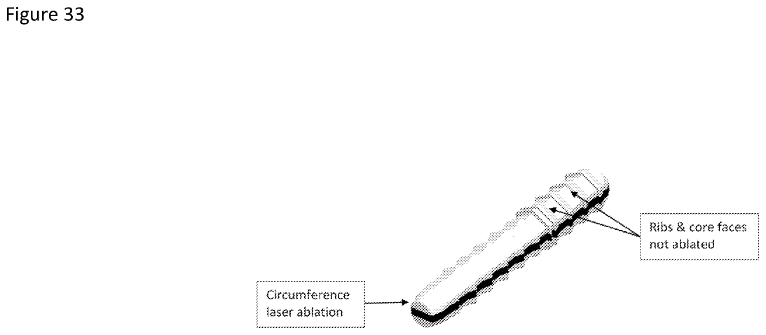

The laser ablation was conducted on all of the circumferential implant to wafer contact line. FIG. 33 depicts an illustration of this surface ablation. An example of such surface treatment is shown in FIG. 19.

The total ablated surface area for this implant was approximately 22.8 mm\hat{ }2. For the defined laser ablation surfaces, the ratio between the ablated surface area to the whole implant surface area is approximately 0.12.

Laser Ablation Application—Direction, Focal Point, Iterations

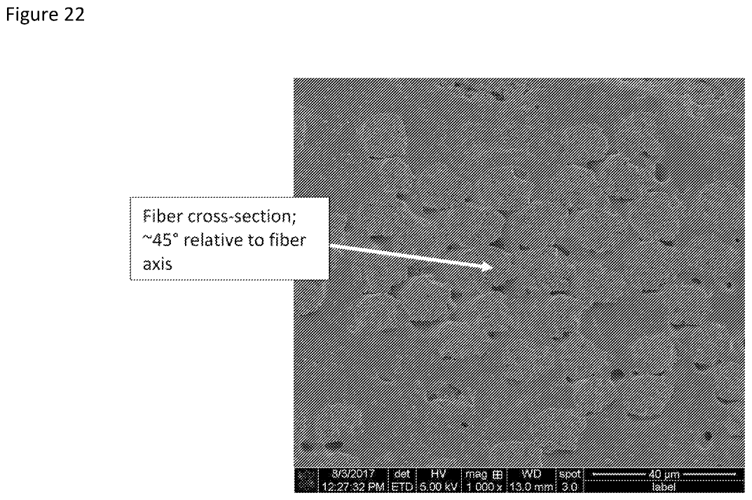

The laser ablation was conducted perpendicular to the implant longitudinal axis. The implant was molded with orientation of layers relative to longitudinal axis of 0° and 25°. This method of laser ablation on the implant core surfaces results in fibers cross-section exposure. An example of such directional fibers exposure is shown in FIGS. 21 and 22.

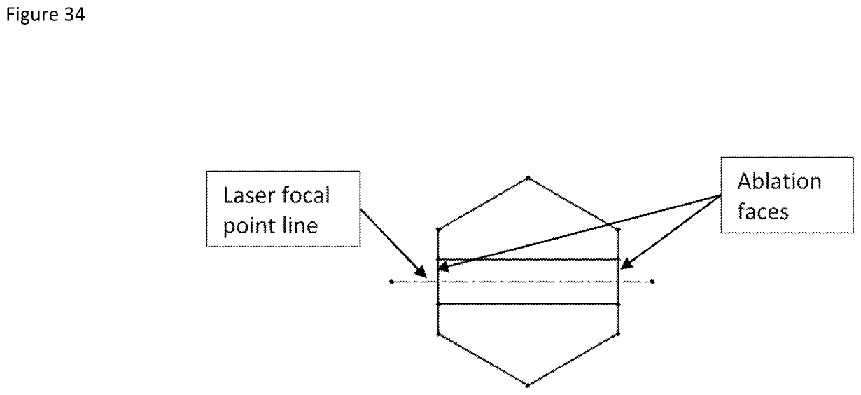

Laser ablation was conducted using multiple laser passes on the wafer to implant circumferential line. The laser focal point was determined at an intermediate point on the line of the wafer prior to treatment, see illustration in FIG. 34.

Results

The result on the implant circumference is a surface with fibers cross-section exposure, with the outer biopolymer layer removed. All other implant surfaces keep their original geometry and original outer layer.

It will be appreciated that various features of the invention which are, for clarity, described in the contexts of separate embodiments may also be provided in combination in a single embodiment. Conversely, various features of the invention which are, for brevity, described in the context of a single embodiment may also be provided separately or in any suitable sub-combination. Various sub-embodiments may be combined in various combinations, even if not explicitly described herein. It will also be appreciated by persons skilled in the art that the present invention is not limited by what has been particularly shown and described hereinabove.

All references cited or described herein are hereby incorporated by reference as if set forth herein to the extent necessary to support the description of the present invention and/or of the appended claims.

Although the invention has been described in conjunction with specific embodiments thereof, it is evident that many alternatives, modifications and variations will be apparent to those skilled in the art. Accordingly, it is intended to additionally embrace all such alternatives, modifications and variations that fall within the spirit and broad scope of the appended claims.

1. An implant with a body composition of bioabsorbable mineral and polymer wherein 10-70% w/w of the body composition comprises mineral material, and wherein said body composition comprises a plurality of fibers, wherein the implant further comprises a surface region comprising polymer; wherein said surface region is ablated to a controlled extent, such that a structure of said fibers is maintained upon ablation of said surface region; wherein the fiber structure is maintained, wherein at least 50, 65, 80, 85, 90, or 95% of fibers retain their geometric structure.

2. The implant of claim 1, wherein the depth of the surface region is in the range of 1-100 micron, 5-50 micron, or 10-30 micron.

3. The implant of claim 1, wherein a fiber diameter is in a range of 2-40 microns, or 4-20 microns.

4. The implant of claim 1, wherein said surface region varies across different cross-sections of the medical implant; and wherein the depth of the surface region is in the range of 1-50 microns in one cross-section of the implant and greater than 50 microns in another cross section; or wherein the depth of the surface region is in the range of 5-50 microns in one cross-section of the implant and greater than 100 microns in another cross section.

5.-6. (canceled)

7. The implant of claim 4, further comprising a plurality of ribs or threads, wherein said surface region is thicker on the implant body comparing to said surface region thickness on the ribs/threads and wherein said ribs/threads are ablated while a remainder of the implant is unablated, or vice versa.

8. (canceled)

9. The implant of claim 1, wherein 10-70% 30-55%, or 15-40% of the medical implant outer surface layer is ablated.

10.-11. (canceled)

12. The implant of claim 1, wherein a depth of the ablation ranges from 1 to 120 microns, 5 to 70 microns, or 5 to 40 microns of the outer surface layer.

13.-16. (canceled)

17. The implant of claim 12, wherein said ablation depth is in a range of 0.1% to 10% or 0.5% to 2.5% of implant wall thickness or implant overall thickness.

18. (canceled)

19. The implant of claim 1, wherein a shape of an area of ablation of the outer surface layer is selected from the group consisting of rectangular, square, circular, arc shaped, diamond, parallelograms, triangular or any combination of said shapes; wherein a shape of an area of ablation of the outer surface layer comprises a line shape of specified width, wherein said surface treated line width is up to 100 microns; or wherein said line shape comprises one or more of continuous solid line, dashed line, dotted line, circumferential line, angled line (any angle from 5 to 85 degrees), helix line (helix angle of 5 to 85 degrees).

20.-36. (canceled)

37. The implant of claim 1, wherein a surface maximum roughness is more than 10 microns

38.-45. (canceled)

46. The implant of claim 1, wherein a resultant surface geometry comprises a portion or an entirety of the ablated surface layer.

47. The implant of claim 46, wherein exposed fibers comprise 20-80%, 35-65%, or 51-70% of the ablated surface layer

48.-51. (canceled)

52. The implant of claim 1, wherein 30-55% w/w or 45-55% w/w of the body composition comprises mineral material.

53. (canceled)

54. The implant of claim 1, wherein the average density of the composite is in the range of 1.1-3.0 g/cm³, 1.2-2.2 g/cm³, or 1.4-1.8 g/cm³

55.-56. (canceled)

57. The implant of claim 1, wherein said body composition comprises a biodegradable polymer; wherein said biodegradable polymer comprises a homopolymer or a copolymer; wherein said copolymer comprises a random copolymer, block copolymer, or graft copolymer; wherein said polymer comprises a linear polymer, a branched polymer, or a dendrimer, of natural or synthetic origin; wherein said polymer comprises lactide, glycolide, caprolactone, valerolactone, carbonates, dioxanones, δ-valerolactone, 1-dioxepanones, ethylene glycol, ethylene oxide, esteramides, γ-hydroxyvalerate, β-hydroxypropionate, alpha-hydroxy acid, hydroxybutyrates, poly (ortho esters), hydroxy alkanoates, tyrosine carbonates, polyimide carbonates, polyimino carbonates, polyurethanes, polyanhydrides, polymer drugs, sugars; starch, cellulose and cellulose derivatives, polysaccharides, collagen, chitosan, fibrin, hyaluronic acid, polypeptides, proteins, poly (amino acids), polylactides (PLA), poly-L-lactide (PLLA), poly-DL-lactide (PDLLA); poly-LD-lactide (PLDLA). polyglycolide (PGA); copolymers of glycolide, glycolide/trimethylene carbonate copolymers (PGA/TMC); lactide/tetramethylglycolide copolymers, lactide/trimethylene carbonate copolymers, lactide/d-valerolactone copolymers, lactide/ε-caprolactone copolymers, L-lactide/DL-lactide copolymers, glycolide/L-lactide copolymers (PGA/PLLA), polylactide-co-glycolide; lactide/glycolide/trimethylene carbonate terpolymers, lactide/glycolide/c:-caprolactone terpolymers, PLA/polyethylene oxide copolymers; polydepsipeptides; unsymmetrically 3,6-substituted poly-I,4-dioxane-2,5-diones; polyhydroxyalkanoates; polyhydroxybutyrates (PHB); PHB/hydroxyvalerate copolymers (PHB/PHV); poly-b-hydroxypropionate (PHPA); poly-p-dioxanone (PDS); poly-d-valerolactone-poly-ε-caprolactone, poly(ε-caprolactone-D L-lactide) copolymers; methylmethacrylate-N-vinyl pyrrolidone copolymers; polyesteramides; polyesters of oxalic acid; polydihydropyrans; polyalkyl-2-cyanoacrylates; polyurethanes (PU); polyvinylalcohol (PV A); polypeptides; poly-b-malic acid (PMLA): poly-b-alkanbic acids; polycarbonates; polyorthoesters; polyphosphates; poly(ester anhydrides); and mixtures thereof; and derivatives, copolymers and mixtures thereof.

58.-60. (canceled)

61. The implant of claim 1, wherein an inherent viscosity (IV) of the polymer matrix alone is in the range of 0.2-6 dl/g, 1.0 to 3.0 dl/g, 1.5 to 2.4 dl/g, or 1.6 to 2.0 dl/g, wherein IV is measured according to a flow time of a polymer solution through a narrow capillary relative to the flow time of the pure solvent through the capillary.

62. The implant of claim 1, wherein said ablation is performed according to an erosive method, irradiation, laser ablation, mechanical brushing, cutting, scraping or chipping.

63.-64. (canceled)

65. The implant of claim 1, wherein said mineral material of said body composition comprises ranges of the following elements, all mol %:
Na₂O: 11.0-19.0 mol. %
CaO: 9.0-14.0 mol. %
MgO: 1.5-8.0 mol. %
B₂O₃: 0.5-3.0 mol. %
Al₂O₃: 0-0.8 mol. %
P₂O₃: 0.1-0.8 mol. %
SiO₂: 67-73 mol. %

66.-67. (canceled)
